SEQUENCE LISTING (5 sequences)

SEQ ID NO: 1 - DNA, 11270 bp, Artificial Sequence
  Suicide plasmid
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccagcgggcaaggaaagccttcaatatgtgcagtggtatgtcaactctatgaagatcagcctgtttacaatggcagggtctttgctctgtgtgacgtttacggcctatgcgttttcgcgctttcggtttaaagggaggaaatacgctttaacgctctttttattgctgcagatgattcctcagttttcagctttaattgccttgtttgtgctggcgcaaatcttgggaatgatcaatagccactggctgctaatcttgctttatatcggcggcctgatcccgatgaatacgtatttgatgaaagggtacatggattccattccgatggatttagacgaaagcgccaagattgacggagccagcagcaccagaatcttcttccagatcattctgccattatcaaaaccgatggcggcagtcgtggccatgaacggctttaccggtccgctcggagattttgtgctgtcctcaaccatattgagaacgcctgaatcatatacattgcccgtcggtctattcaatttagtgaatgatgtcatgggggccagctatacgacatttgcggccggagccctgcttatcagcataccggttgccgtcatctttattatgctgcaaaagaattttgtgtccggattaaccgcaggcggaacgaagggctaagagaacaaggaggagaatgtgatgtcaaagcttgaaaaaacgcacgtaacaaaagcaaaatttatgctccatgggggagactacaaccccgatcagtggctggatcggcccgatattttagctgacgatatcaaactgatgaagctttctcatacgaatacgttttctgtcggcaccagtgaattcgagctcggtacctaccgttcgtataatgtatgctatacgaagttattacgcgttaacccgggcccgcggatgcatatgatcagatcctttaactctggcaaccctcaaaattgaatgagacatgctacacctccggataataaatatatataaacgtatatagatttcataaagtctaacacactagacttatttacttcgtaattaagtcgttaaaccgtgtgctctacgaccaaaactataaaacctttaagaactttctttttttacaagaaaaaagaaattagataaatctctcatatcttttattcaataatcgcatccgattgcagtataaatttaacgatcactcatcatgttcatatttatcagagctcgtgctataattatactaattttataaggaggaaaaaatatgggcatttttagtatttttgtaatcagcacagttcattatcaaccaaacaaaaaataagtggttataatgaatcgttaataagcaaaattcatataaccaaattaaagagggttataatgaacgagaaaaatataaaacacagtcaaaactttattacttcaaaacataatatagataaaataatgacaaatataagattaaatgaacatgataatatctttgaaatcggctcaggaaaaggccattttacccttgaattagtaaagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatatggtaatataccttataacataagtacggatataatacgcaaaattgtttttgatagtatagctaatgagatttatttaatcgtggaatacgggtttgctaaaagattattaaatacaaaacgctcattggcattacttttaatggcagaagttgatatttctatattaagtatggttccaagagaatattttcatcctaaacctaaagtgaatagctcacttatcagattaagtagaaaaaaatcaagaatatcacacaaagataaacaaaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtgcctattttttgtgaatcgattataacttcgtataatgtatgctatacgaacggtaggatcctctagagtcgacctgcaggcattttacatttttagaaatgggcgtgaaaaaaagcgcgcgattatgtaaaatataaagattaactaataaggaggacaaacatggcacaatctaaactgtatccggttgtgatggcgggcggatctggatcaagactgtggccgctttcacgcgttctttacccgaaacaatttctgtgtctgaaaggagatttaacaatgctgcagacaacaatttgcagattaaatggcgtggaatgtgaatctccggtcgttatttgcaacgaacagcatagatttatcgtcgctgaacaacttcgccagctgaacaaactgacagaaaacatcatcctggaaccggccggcagaaatacagcaccggcgattgctcttgcagcgttagctgccaaacgccattcaccggaaagcgatccgcttatgttagtgctggcagcggatcatgtcattgcggatgaagatgcttttagagctgccgttcgcaacgctatgccgtatgccgaagcaggcaaacttgtgacatttggaattgtcccggatttaccggaaacaggctatggatatatcagacgcggcgaagtgtcagcgggagaacaagatatggttgcctttgaagtggcacagtttgtcgaaaaaccgaatctggaaacagcgcaagcgtatgttgcttcaggcgaatattattggaacagcggaatgtttctttttagagccggccgctatctggaagaacttaaaaaatacagaccggatattttagatgcatgtgaaaaagcgatgtcagctgtcgatccggatttaaactttatcagagttgatgaagaagcctttctggcatgcccggaagaaagcgttgattatgcagtgatggaacgcacagccgatgcagtggtcgttccgatggatgcgggctggtctgatgttggatcatggtcaagcctttgggaaattagcgcgcatacagctgaaggcaatgtttgtcatggagatgtgatcaaccataaaacagaaaacagctatgtttacgcggaatctggcctggtgacaacagtcggagttaaagatttagtggtcgttcagacaaaagatgcggtgttaattgctgatagaaacgccgtgcaagatgttaagaaagttgtcgaacagatcaaagcagatggaagacatgaacatagagtccatcgcgaagtttatcgcccgtggggaaaatatgatagcattgatgcgggcgatcgctatcaagtcaaacgcatcacagttaaaccgggcgaaggactgtctgttcagatgcatcatcatcgcgcagaacattgggttgtggtcgccggcacagcaaaagtgacaattgatggcgatattaaactgcttggagaaaacgaatctatttatatcccgttaggagctacacattgcctggaaaacccgggcaaaattccgcttgatttaatcgaagtcagaagcggatcttatcttgaagaagatgatgttgtgcgctttgccgatagatatggccgcgtttaagctcttaaggaggattttagaatgaaaaaacttacatgctttaaagcgtatgatattcgcggcaaacttggagaagaattaaacgaagatattgcttggagaatcggccgcgcctatggagaatttctgaaaccgaaaacaatcgtgctgggcggagatgtcagacttacatcagaaacacttaaactggctctggccaaaggcctgcaagatgctggagttgatgtgcttgatattggcatgagcggaacagaagaaatctattttgccacatttcatttaggagtcgatggcggaatcgaagttacagcaagccataatccgatggattataacggaatgaaactggttagagaaggcgcaagaccgatttctggcgatacaggattaagagatgtgcagcgcctggcagaagcgaacgattttccgccggtcgatgaaacaaaaagaggacgctatcaacagattaatttacgcgatgcgtatgttgatcatctttttggctacatcaacgttaaaaacctgacaccgcttaaactggtgatcaattctggcaacggagcagcgggaccggttgtggatgctattgaagccagatttaaagcacttggcgcaccggttgaattaatcaaagtgcataatacaccggatggcaattttccgaacggcattccgaacccgctgcttccggaatgcagagatgatacacgcaatgcggttattaaacatggcgctgatatgggaatcgcctttgatggagattttgatcgctgttttctttttgatgaaaaaggccaatttatcgaaggatactacatcgtgggcttactggctgaagcatttctggagaaaaatccgggagcgaaaattatccatgatccgagactttcttggaatacagtcgatgtcgttacagctgccggcggaacaccggttatgtcaaaaacaggccatgcttttatcaaagaaagaatgcgcaaagaagatgcaatctatggcggagaaatgagcgcgcatcattactttagagattttgcttactgcgattctggcatgatcccgtggcttttagtcgcggaactggtttgtcttaaagataaaacactgggagaactggtcagagatcgcatggcagcgtttccggcttcaggcgaaattaatagcaaactggcacaaccggtggaagcgatcaacagagtcgaacagcatttttcacgcgaagcactggcggttgatagaacagatggaattagcatgacatttgccgattggagatttaatcttcgcacatctaacacagaaccggtggtcagacttaacgttgaatcacgcggcgatgtgccgttaatggaagcaagaacacgcacactgcttacattactgaatgaataaaaaggaggaactactatgacaacagaaccgctgtttttcaaaccggtctttaaagaaagaatttggggcggaacagctctggccgattttggctatacaatcccgtctcaacgcacaggagaatgctgggcttttgcagcgcatcaaaatggccagagcgttgtgcagaacggcatgtacaaaggatttacactgtctgaactgtgggaacatcatagacatctttttggccaattagaaggagatcgctttccgctgcttacaaaaattttagatgctgatcaggatctgtcagttcaggttcatccgaatgatgaatatgccaatatccatgaaaacggcgaacttggaaaaacagaatgctggtatattatcgattgtcagaaagatgctgaaattatctatggccataacgccacaacaaaagaagaattaacaacaatgatcgaaagaggagaatgggatgaattactgagacgcgtgaaagtcaaaccgggcgatttcttttatgtcccgtcaggaacagttcatgcaattggcaaaggaatccttgcgttagaaacacaacagaatagcgatacaacatacagactgtacgattacgatcgcaaagatgcagaaggcaaacttagagaactgcatcttaaaaaatcaatcgaagttatcgaagtgccgagcattccggaacgccatacagttcatcatgaacaaatcgaagatttactgacaacaacactgatcgaatgcgcatattttagcgtgggcaaatggaacctttcaggaagcgcgtctttaaaacaacagaaaccgtttctgcttatttctgtgatcgaaggcgaaggacgcatgatttcaggcgaatatgtctatccgtttaagaaaggcgatcacatgttactgccgtatggcctgggagaatttaaacttgaaggatatgcggaatgtatcgtttctcatctttaagcgaggcaggatcaggacaatgattatatttttgtcatgaatttcacggaagaaaaacagctggtcacgtttgatcagagtgtgaaggacataatgacaggagacatattgtcaggcgacctgacgatggaaaagtatgaagtgagaattgtcgtaaacacacattaggctgatgctccgctcgatatgggcggattcttttttctatagaatgaaaacgcttgctaagtcttggggggatgaaatcatgaaaagcaaagtgaaaatgttctttgcggctgccatcgtgtggagtgcatgtagttcaacaggatatgccgctgccattgagaaggagaagcacgtgtcagagcttcgggcagaggatctttttgttaaaaaagtagaggggatgaacaaggattttatcaaaggggcagatgtatccagcgttattgctttggaaaacagcggtgtcaccttttacaatacaaacggaaaacgccaggatatctttacaactttaaaacaggctggggtcaactatgttcgcgtccgcatctggaatcacccgtatgattcaaatggcaacgggtatggcgggggaaacaatgatgttcaaaaagccatcgaaatcggaaaaagagcgacagcgaacggaatgaaggtgctggccgactttcactactctgatttctgggccgatccagcgaaacaaaaggtgcccaaagcctgggcgaatctcagctttgaagcaaaaaaagcaaagctctatgagtatacgaaacaaagcctgcaaaagatgatcaaggaaggcgtgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 2 - DNA, 12427 bp, Artificial Sequence
  expression plasmid
ttaagttattggtatgactggttttaagcgcaaaaaaagttgctttttcgtacctattaatgtatcgttttagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaagaccacattaaaaaatgtggtcttttgtgtttttttaaaggatttgagcgtagcgaaaaatccttttctttcttatcttgataataagggtaactattgccgatcgtccattccgacagcatcgccagtcactatggcgtgctgctagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcaggccttaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatcaaaatcgtctccctccgtttgaatatttgattgatcgtaaccagatgaagcactctttccactatccctacagtgttatggcttgaacaatcacgaaacaataattggtacgtacgatctttcagccgactcaaacatcaaatcttacaaatgtagtctttgaaagtattacatatgtaagatttaaatgcaaccgttttttcggaaggaaatgatgacctcgtttccaccggaattagcttggtaccaaaggaggtaaggatcactagaaaattttttaaaaaatctcttgacattggaagggagatatgttattataagaattgcggaattgtgagcggataacaattcccatatagattaactaataaggaggacaaacatgggttctatcatcagacttcagggtggtttaggcaatcaactgtttcaatttagctttggttatgctctttcaaaaatcaatggcacaccgctgtactttgatatttcacattacgcagaaaacgatgatcatggcggatatagactgaataaccttcaaatcccggaagaatatcttcagtactacacaccgaaaatcaacaacatctacaaactgcttgtcagaggcagccgcttatatccggatatttttctgtttcttggcttttgcaacgaatttcatgcgtatggctatgattttgaatacatcgcgcaaaaatggaaaagcaaaaaatacatcggatactggcagtctgaacatttctttcataaacatattctggatctgaaagaatttttcatcccgaaaaacgttagcgaacaagctaacttactggcagcgaaaatcttagaatctcagtcaagcctgtcaattcatatcagacgcggcgattacatcaaaaacaaaacagccacactgacacatggagtttgctctcttgaatactacaagaaagcactgaacaaaatcagagatttagccatgattcgcgatgtgtttatcttttctgatgatattttctggtgtaaagaaaacatcgaaacacttctgagcaaaaaatacaacatctactactcagaagatttaagccaagaagaagatttatggctgatgtctctggctaatcatcatattatcgccaactcttcattttcatggtggggcgcatatcttggaagctctgcgagccagattgttatctatccgacaccgtggtatgatattacaccgaaaaacacatacattcctatcgtgaaccattggatcaacgtggacaaacattcctcctgctaaggagaaaagctagcgattaactaataaggaggaactgaaatgtcaaaagtggcgcttattacaggcgtcacaggacaagatggcagctatttagctgaatttctgcttgaaaaaggatatgaagtgcatggcattaaaagacgcgcatcatcatttaacacagaaagagtcgatcatatctatcaagatccgcatacatgcaacccgaaatttcatctgcattatggcgatctgagcgatacatctaacctgacaagaattttacgcgaagtccagccggatgaagtttataacctgggcgcgatgtctcatgtcgctgtttcatttgaaagcccggaatatacagccgatgtcgatgcaatgggaacacttagattactggaagccattcgctttttaggcctggaaaagaaaacacgcttttatcaggcatctacatcagaactgtatggacttgttcaagaaatcccgcagaaagaaacaacaccgttttatccgagaagcccgtatgcggtcgctaaactttacgcgtactggatcacagttaattatcgcgaatcttatggaatgtatgcttgcaatggcatcttatttaaccatgaatcaccgagacgcggagaaacatttgttacaagaaaaatcacacgcgccattgcaaatatcgcccaaggactggaaagctgtctttatttaggcaacatggattctcttagagattggggccatgcaaaagattacgttaaaatgcaatggatgatgttacaacaggaacagccggaagattttgtgatcgccacaggagtgcaatattcagtcagacagtttgttgaaatggcagcggctcagcttggcattaaactgcgctttgaaggcacaggagtggaagaaaaaggaatcgttgtgagcgttacaggccatgatgcgccgggagtgaaaccgggcgatgtgattatcgccgtcgatccgagatattttcgcccggcagaagtcgaaacacttttaggagatccgacaaaagcgcatgaaaaactgggctggaaaccggaaattacactgagagaaatggttagcgaaatggtggctaacgatttagaagccgcgaaaaaacattctctgcttaaatcacatggctatgatgtggcgatcgctctggaatcttaaaaaggaggaactactatgagcaaacaaagagtttttatcgctggccatcgcggaatggtgggctctgccattagacgccaattagaacagagaggagatgtggaacttgtcttaagaacacgcgatgaactgaatctgcttgattcaagagctgtccatgatttctttgccagcgaacgcatcgatcaggtttatcttgcagcggctaaagttggcggaattgtggcaaataacacatatccggcggattttatctaccaaaacatgatgatcgaatctaacattatccatgccgcacatcagaatgatgttaacaaactgctgtttctgggatcaagctgcatctatccgaaacttgcaaaacaaccgatggcggaatcagaacttttacagggcacactggaaccgacaaatgaaccgtatgcaattgcgaaaatcgcgggaatcaaactttgtgaatcttacaacagacaatacggcagagattatcgctcagtgatgccgacaaacttatatggaccgcatgataattttcatccgtctaactcacatgtcattccggcactgcttagacgctttcatgaagctacagcccagaacgcgccggatgttgtggtctggggctcaggaacaccgatgcgcgaatttctgcatgttgatgatatggcggctgccagcatccatgtgatggaacttgctcatgaagtctggttagaaaatacacaaccgatgcttagccatattaacgtcggcacaggagttgattgcacaatcagagaattagctcagacaattgccaaagttgtgggatataaaggccgcgtcgtttttgatgcctctaaaccggatggcacaccgagaaaattactggatgtgacacgcctgcatcaacttggatggtatcatgaaattagcttagaagcaggcctggcgtctacataccaatggtttttagaaaaccaggatagatttcgcggcggatcataaagtgatagcggtaccattataggtaagagaggaatgtacacatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaaggatccatgtctagagtcgacgtccccggggcagcccgcctaatgagcgggcttttttcacgtcccaggcatcaaataaaacgaaaggctcagtcgaaagactgggcctttcgttttatctgttgtttgtcggtgaacgctctctactagagtcacactggctcaccttcgggtgggcctttctgcgtttatacccggggcagcccgcctaatgagcgggcttttttcacgtcacgcgtccatggagatctttgtctgcaactgaaaagtttataccttacctggaacaaatggttgaaacatacgaggctaatatcggcttattaggaatagtccctgtactaataaaatcaggtggatcagttgatcagtatattttggacgaagctcggaaagaatttggagatgacttgcttaattccacaattaaattaagggaaagaataaagcgatttgatgttcaaggaatcacggaagaagatactcatgataaagaagctctaaaactattcaataaccttacaatggaattgatcgaaagggtggaaggttaatggtacgaaaattaggggatctacctagaaagccacaaggcgataggtcaagcttaaagaacccttacatggatcttacagattctgaaagtaaagaaacaacagaggttaaacaaacagaaccaaaaagaaaaaaagcattgttgaaaacaatgaaagttgatgtttcaatccataataagattaaatcgctgcacgaaattctggcagcatccgaagggaattcatattacttagaggatactattgagagagctattgataagatggttgagacattacctgagagccaaaaaactttttatgaatatgaattaaaaaaaagaaccaacaaaggctgagacagactccaaacgagtctgtttttttaaaaaaaatattaggagcattgaatatatattagagaattaagaaagacatgggaataaaaatattttaaatccagtaaaaatatgataagattatttcagaatatgaagaactctgtttgtttttgatgaaaaaacaaacaaaaaaaatccacctaacggaatctcaatttaactaacagcggccaaactgagaagttaaatttgagaaggggaaaaggcggatttatacttgtatttaactatctccattttaacattttattaaaccccatacaagtgaaaatcctcttttacactgttcctttaggtgatcgcggagggacattatgagtgaagtaaacctaaaaggaaatacagatgaattagtgtattatcgacagcaaaccactggaaataaaatcgccaggaagagaatcaaaaaagggaaagaagaagtttattatgttgctgaaacggaagagaagatatggacagaagagcaaataaaaaacttttctttagacaaatttggtacgcatataccttacatagaaggtcattatacaatcttaaataattacttctttgatttttggggctattttttaggtgctgaaggaattgcgctctatgctcacctaactcgttatgcatacggcagcaaagacttttgctttcctagtctacaaacaatcgctaaaaaaatggacaagactcctgttacagttagaggctacttgaaactgcttgaaaggtacggttttatttggaaggtaaacgtccgtaataaaaccaaggataacacagaggaatccccgatttttaagattagacgtaaggttcctttgctttcagaagaacttttaaatggaaaccctaatattgaaattccagatgacgaggaagcacatgtaaagaaggctttaaaaaaggaaaaagagggtcttccaaaggttttgaaaaaagagcacgatgaatttgttaaaaaaatgatggatgagtcagaaacaattaatattccagaggccttacaatatgacacaatgtatgaagatatactcagtaaaggagaaattcgaaaagaaatcaaaaaacaaatacctaatcctacaacatcttttgagagtatatcaatgacaactgaagaggaaaaagtcgacagtactttaaaaagcgaaatgcaaaatcgtgtctctaagccttcttttgatacctggtttaaaaacactaagatcaaaattgaaaataaaaattgtttattacttgtaccgagtgaatttgcatttgaatggattaagaaaagatatttagaaacaattaaaacagtccttgaagaagctggatatgttttcgaaaaaatcgaactaagaaaagtgcaataaactgctgaagtatttcagcagttttttttatttagaaatagtgaaaaaaatataatcagggaggtatcaatatttaatgagtactgatttaaatttatttagactggaattaataattaacacgtagactaattaaaatttaatgagggataaagaggatacaaaaatattaatttcaatccctattaaattttaacaagggggggattaaaatttaattagaggtttatccacaagaaaagaccctaataaaatttttactagggttataacactgattaatttcttaatgggggagggattaaaatttaatgacaaagaaaacaatcttttaagaaaagcttttaaaagataataataaaaagagctttgcgattaagcaaaactctttactttttcattgacattatcaaattcatcgatttcaaattgttgttgtatcataaagttaattctgttttgcacaaccttttcaggaatataaaacacatctgaggcttgttttataaactcagggtcgctaaagtcaatgtaacgtagcatatgatatggtatagcttccacccaagttagcctttctgcttcttctgaatgtttttcatatacttccatgggtatctctaaatgattttcctcatgtagcaaggtatgagcaaaaagtttatggaattgatagttcctctctttttcttcaacttttttatctaaaacaaacactttaacatctgagtcaatgtaagcataagatgtttttccagtcataatttcaatcccaaatcttttagacagaaattctggacgtaaatcttttggtgaaagaatttttttatgtagcaatatatccgatacagcaccttctaaaagcgttggtgaatagggcattttacctatctcctctcattttgtggaataaaaatagtcatattcgtccatctacctatcctattatcgaacagttgaactttttaatcaaggatcagtcctttttttcattattcttaaactgtgctcttaactttaacaactcgatttgtttttccagatctcgagggtaactagcctcgccgatcccgcaagaggcccggcagtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcatgc

SEQ ID NO: 3 - DNA, 12330 bp, Artificial Sequence
  integration vector
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgacccgacatccggcgttctcatggcggtgcttgccgccagcggtattccgtatgtcaagtggctgcggtttatggtgccgcttgctctgatttggttcttgatcgggcttgtctttatcgtgatcggagtcatgatcaattgggggccgttttaacgattgctgcccgccggcttgtacggcgggcttttgagttattcattgcagaagcgcaggctgttattgtaacatgtaagccataagccattcgtaaaagtgcgggaggaaggtcatgaataatctgcgtaatagactttcaggcgtgaatgggaaaaataagagagtaaaagaaaaagaacaaaaaatctggtcggagattgggatgatagcgggagcatttgcgctgcttgatgtgatcatccgcggcattatgtttgaatttccgtttaaagaatgggctgcaagccttgtgtttttgttcatcattatcttatattactgcatcagggctgcggcatccggaatgctcatgccgagaatagacaccaaagaagaactgcaaaaacgggtgaagcagcagcgaatagaatcaattgcggtcgcctttgcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaccagtgaattcgagctcggtacctaccgttcgtataatgtatgctatacgaagttatgataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaatataacttcgtataatgtatgctatacgaacggtaggatcctctagagtcgacctgcaggcattttacatttttagaaatgggcgtgaaaaaaagcgcgcgattatgtaaaatataaagattaactaataaggaggacaaacatgggttctatcatcagacttcagggtggtttaggcaatcaactgtttcaatttagctttggttatgctctttcaaaaatcaatggcacaccgctgtactttgatatttcacattacgcagaaaacgatgatcatggcggatatagactgaataaccttcaaatcccggaagaatatcttcagtactacacaccgaaaatcaacaacatctacaaactgcttgtcagaggcagccgcttatatccggatatttttctgtttcttggcttttgcaacgaatttcatgcgtatggctatgattttgaatacatcgcgcaaaaatggaaaagcaaaaaatacatcggatactggcagtctgaacatttctttcataaacatattctggatctgaaagaatttttcatcccgaaaaacgttagcgaacaagctaacttactggcagcgaaaatcttagaatctcagtcaagcctgtcaattcatatcagacgcggcgattacatcaaaaacaaaacagccacactgacacatggagtttgctctcttgaatactacaagaaagcactgaacaaaatcagagatttagccatgattcgcgatgtgtttatcttttctgatgatattttctggtgtaaagaaaacatcgaaacacttctgagcaaaaaatacaacatctactactcagaagatttaagccaagaagaagatttatggctgatgtctctggctaatcatcatattatcgccaactcttcattttcatggtggggcgcatatcttggaagctctgcgagccagattgttatctatccgacaccgtggtatgatattacaccgaaaaacacatacattcctatcgtgaaccattggatcaacgtggacaaacattcctcctgctaaagtgatagcggtaccattataggtaagagaggaatgtacacatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaaagtcaatgtatgaatggatacgggatatgaatcaataagtacgtgaaagagaaaagcaacccagatatgatagggaacttttctctttcttgttttacattgaatctttacaatcctattgatataatctaagctagtgtattttgcgtttaatagtggagaaaagctagcgattaactaataaggaggaactgaaatgtcaaaagtggcgcttattacaggcgtcacaggacaagatggcagctatttagctgaatttctgcttgaaaaaggatatgaagtgcatggcattaaaagacgcgcatcatcatttaacacagaaagagtcgatcatatctatcaagatccgcatacatgcaacccgaaatttcatctgcattatggcgatctgagcgatacatctaacctgacaagaattttacgcgaagtccagccggatgaagtttataacctgggcgcgatgtctcatgtcgctgtttcatttgaaagcccggaatatacagccgatgtcgatgcaatgggaacacttagattactggaagccattcgctttttaggcctggaaaagaaaacacgcttttatcaggcatctacatcagaactgtatggacttgttcaagaaatcccgcagaaagaaacaacaccgttttatccgagaagcccgtatgcggtcgctaaactttacgcgtactggatcacagttaattatcgcgaatcttatggaatgtatgcttgcaatggcatcttatttaaccatgaatcaccgagacgcggagaaacatttgttacaagaaaaatcacacgcgccattgcaaatatcgcccaaggactggaaagctgtctttatttaggcaacatggattctcttagagattggggccatgcaaaagattacgttaaaatgcaatggatgatgttacaacaggaacagccggaagattttgtgatcgccacaggagtgcaatattcagtcagacagtttgttgaaatggcagcggctcagcttggcattaaactgcgctttgaaggcacaggagtggaagaaaaaggaatcgttgtgagcgttacaggccatgatgcgccgggagtgaaaccgggcgatgtgattatcgccgtcgatccgagatattttcgcccggcagaagtcgaaacacttttaggagatccgacaaaagcgcatgaaaaactgggctggaaaccggaaattacactgagagaaatggttagcgaaatggtggctaacgatttagaagccgcgaaaaaacattctctgcttaaatcacatggctatgatgtggcgatcgctctggaatcttaaaaaggaggaactactatgagcaaacaaagagtttttatcgctggccatcgcggaatggtgggctctgccattagacgccaattagaacagagaggagatgtggaacttgtcttaagaacacgcgatgaactgaatctgcttgattcaagagctgtccatgatttctttgccagcgaacgcatcgatcaggtttatcttgcagcggctaaagttggcggaattgtggcaaataacacatatccggcggattttatctaccaaaacatgatgatcgaatctaacattatccatgccgcacatcagaatgatgttaacaaactgctgtttctgggatcaagctgcatctatccgaaacttgcaaaacaaccgatggcggaatcagaacttttacagggcacactggaaccgacaaatgaaccgtatgcaattgcgaaaatcgcgggaatcaaactttgtgaatcttacaacagacaatacggcagagattatcgctcagtgatgccgacaaacttatatggaccgcatgataattttcatccgtctaactcacatgtcattccggcactgcttagacgctttcatgaagctacagcccagaacgcgccggatgttgtggtctggggctcaggaacaccgatgcgcgaatttctgcatgttgatgatatggcggctgccagcatccatgtgatggaacttgctcatgaagtctggttagaaaatacacaaccgatgcttagccatattaacgtcggcacaggagttgattgcacaatcagagaattagctcagacaattgccaaagttgtgggatataaaggccgcgtcgtttttgatgcctctaaaccggatggcacaccgagaaaattactggatgtgacacgcctgcatcaacttggatggtatcatgaaattagcttagaagcaggcctggcgtctacataccaatggtttttagaaaaccaggatagatttcgcggcggatcataaggatccatgtctagagtcgacgtccccggggcagcccgcctaatgagcgggcttttttcacgtcccaggcatcaaataaaacgaaaggctcagtcgaaagactgggcctttcgttttatctgttgtttgtcggtgaacgctctctactagagtcacactggctcaccttcgggtgggcctttctgcgtttatagaattcatattacttagaggatactattgagagagctattgataagatggttgagacattacctgagagccaaaaaactttttatgaatatgaattaaaaaaaagaaccaacaaaggctgagacagactccgggcaaggctagacgggacttaccgaaagaaaccatcaatgatggtttcttttttgttcataaatcagacaaaacttttctcttgcaaaagtttgtgaagtgttgcacaatataaatgtgaaatacttcacaaacaaaaagacatcaaagagaaacataccctggaaggatgattaatgatgaacaaacatgtaaataaagtagctttaatcggagcgggttttgttggaagcagttatgcatttgcgttaattaaccaaggaatcacagatgagcttgtggtcattgatgtaaataaagaaaaagcaatgggcgatgtgatggatttaaaccacggaaaggcgtttgcgccacaaccggtcaaaacatcttacggaacatatgaagactgcaaggatgctgatattgtctgcatttgcgccggagcaaaccaaaaacctggtgagacacgccttgaattagtagaaaagaacttgaagattttcaaaggcatcgttagtgaagtcatggcgagcggatttgacggcattttcttagtcgcgacaaatccggttgatatcctgacttacgcaacatggaaattcagcggcctgccaaaagagcgggtgattggaagcggcacaacacttgattctgcgagattccgtttcatgctgagcgaatactttggcgcagcgcctcaaaacgtacacgcgcatattatcggagagcacggcgacacagagcttcctgtttggagccacgcgaatgtcggcggtgtgccggtcagtgaactcgttgagaaaaacgatgcgtacaaacaagaggagctggaccaaattgtagatgatgtgaaaaacgcagcttaccatatcattgagaaaaaaggcgcgacttattatggggttgcgatgagtcttgctcgcattacaaaagccattcttcataatgaaaacagcatattaactgtcagcacatatttggacgggcaatacggtgcagatgacgtgtacatcggtgtcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 4 - DNA, 4254 bp, Artificial Sequence
  integration cassette
gcaggctgttattgtaacatgtaagccataagccattcgtaaaagtgcgggaggaaggtcatgaataatctgcgtaatagactttcaggcgtgaatgggaaaaataagagagtaaaagaaaaagaacaaaaaatctggtcggagattgggatgatagcgggagcatttgcgctgcttgatgtgatcatccgcggcattatgtttgaatttccgtttaaagaatgggctgcaagccttgtgtttttgttcatcattatcttatattactgcatcagggctgcggcatccggaatgctcatgccgagaatagacaccaaagaagaactgcaaaaacgggtgaagcagcagcgaatagaatcaattgcggtcgcctttgcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggccagtgaattcgagctcggtacctaccgttcgtataatgtatgctatacgaagttatgataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaatataacttcgtataatgtatgctatacgaacggtaggatcctctagagtcgacctgcaggcattttacatttttagaaatgggcgtgaaaaaaagcgcgcgattatgtaaaatataaagtgatagcggtaccattataggtaagagaggaatgtacacatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaagcaatcaatgtcggatgccagcctggctttgattacgtgctaaatggtttatataatgactcgggcttaagcggttctcttccccattgagggcaaggctagacgggacttaccgaaagaaaccatcaatgatggtttcttttttgttcataaatcagacaaaacttttctcttgcaaaagtttgtgaagtgttgcacaatataaatgtgaaatacttcacaaacaaaaagacatcaaagagaaacataccctggaaggatgattaatgatgaacaaacatgtaaataaagtagctttaatcggagcgggttttgttggaagcagttatgcatttgcgttaattaaccaaggaatcacagatgagcttgtggtcattgatgtaaataaagaaaaagcaatgggcgatgtgatggatttaaaccacggaaaggcgtttgcgccacaaccggtcaaaacatcttacggaacatatgaagactgcaaggatgctgatattgtctgcatttgcgccggagcaaaccaaaaacctggtgagacacgccttgaattagtagaaaagaacttgaagattttcaaaggcatcgttagtgaagtcatggcgagcggatttgacggcattttcttagtcgcgacaaatccggttgatatcctgacttacgcaacatggaaattcagcggcctgccaaaagagcgggtgattggaagcggcacaacacttgattctgcgagattccgtttcatgctgagcgaatactttggcgcagcgcctcaaaacgtacacgcgcatattatcggagagcacggcgacacagagcttcctgtttggagccacgcgaatgtcggcggtgtgccggtcagtgaactcgttgagaaaaacgatgcg

SEQ ID NO: 5 - DNA, 10340 bp, Artificial Sequence
  expression plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgccttcaatccttttaataacaattatagcatctaatcttcaacaaactggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttagtcacttataactttaacagatggggtttttctgtgcaaccaattttaagggttttcaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagccgtctgtacgttccttaagattttacatttttagaaatgggcgtgaaaaaaagcgcgcgattatgtaaaatataaagattaactaataaggaggacaaacatgcaaaaactgctttctctgccgtcaaatcttgtccagtcttttcatgaacttgaaagagttaaccgcacagattggttttgcacatcagatccggttggcaaaaaactgggctcaggcggaggcacaagctggttactggaagaatgttacaacgaatacagcgatggagcgacatttggcgaatggctggaaaaagaaaaacgcattctgttacatgctggaggccaaagcagacgccttccgggatatgccccgtctggcaaaatcttaacaccggtcccggtttttagatgggaaagaggacaacatttaggccagaacctgcttagcttacaactgccgctttatgaaaaaattatgtctctggctccggataaactgcatacactgattgcctctggcgatgtgtatatccgcagcgaaaaaccgcttcagtctatcccggaagctgatgttgtgtgctatggattatgggtcgatccgtctctggcaacacatcatggcgtttttgcgtcagatagaaaacatccggaacaactggattttatgctgcagaaaccgtcacttgcagaattagaaagcctgtctaaaacacatttatttctgatggatattggcatctggttactgtcagatagagcggtcgaaattctgatgaaacgcagccataaagaatcaagcgaagaacttaaatactacgatctgtactcagattttggacttgctttaggcacacatccgcgcattgaagatgaagaagtgaatacacttagcgtcgcaatcctgcctcttccgggaggcgaattttaccattacggaacatcaaaagaactgatttcttcaacacttagcgttcaaaacaaagtgtatgatcagagacgcatcatgcatagaaaagtgaaaccgaatccggcaatgtttgtccaaaacgcggtcgttcgcattccgctttgcgctgaaaatgccgatttatggattgaaaacagccatatcggaccgaaatggaaaatcgcatcaagacatattatcacaggcgtgccggaaaatgattggtcacttgcagtcccggcgggagtgtgtgtcgatgtggtcccgatgggagataaaggctttgttgcgagaccgtatggattagatgatgtgtttaaaggcgatctgcgcgatagcaaaacaacacttacaggcattccgtttggcgaatggatgtctaaaagaggattatcatacacagatttaaaaggccgcacagatgatctgcaggctgtttcagtgtttccgatggtgaacagcgtcgaagaattaggcctggtccttagatggatgctttctgaaccggaattagaagaaggaaaaaatatttggctgcgctctgaacatttttcagcagatgaaatctcagctggcgccaaccttaaaagactgtacgcgcaaagagaagaatttcgcaaaggaaattggaaagcacttgcggttaaccatgaaaaatcagtgttttatcagctggatctggcagatgcagcggaagattttgttcgccttggcttagatatgccggaacttttaccggaagatgctctgcaaatgtcacgcattcataatcgcatgcttagagcccgcatcctgaaacttgatggaaaagattatagaccggaagaacaggctgcctttgatctgcttagagatggattactggatggcattagcaaccgcaaatctacaccgaaactggatgtctattctgatcaaattgtttggggcagatcaccggtgcgcatcgatatggcaggcggctggacagatacaccgccgtatagcctttattctggaggcaatgttgtgaaccttgcgattgaattaaatggccaaccgccgcttcaggtctacgttaaaccgtgcaaagattttcatatcgttctgagatcaatcgatatgggagctatggaaatcgtgagcacatttgatgaactgcaagattacaagaaaattggctcaccgtttagcatcccgaaagcagcgttaagcctggccggatttgctccggccttttctgctgtcagctatgcatcactggaagaacagcttaaagattttggcgcaggaattgaagttacacttttagctgccatcccggcgggctctggattaggcacaagctctattctggcttcaacagtgcttggagccatcaatgatttttgcggcctggcatgggataaaaacgaaatttgtcaaagaacacttgtcttagaacagctgcttacaacaggcggcggctggcaagatcagtatggcggagtcctgcaaggagttaaactgctgcagacagaagctggctttgcccagtcaccgttagttcgctggctgccggatcatctttttacacatccggaatacaaagattgtcatcttttatactacacaggaattacaagaacagcaaaaggcattttagcggaaatcgtgtcaagcatgtttctgaatagctcactgcatcttaacctgcttagcgaaatgaaagcacatgcgcttgatatgaatgaagctattcaaagaggctcttttgtcgaatttggacgcttagttggcaaaacatgggaacagaataaagccctggatagcggaacaaacccgccggcagtggaagcgatcatcgatctgatcaaagattacacactgggatacaaactgcctggagcaggcggcggcggctatctgtatatggttgcgaaagatccgcaagcagctgtgagaattagaaaaatcctgacagaaaatgctccgaacccgagagcccgctttgtggaaatgacactttcagataaaggatttcaggtctcaagaagctaagctcttaaggaggattttagaatgggttctatcatcagacttcagggtggtttaggcaatcaactgtttcaatttagctttggttatgctctttcaaaaatcaatggcacaccgctgtactttgatatttcacattacgcagaaaacgatgatcatggcggatatagactgaataaccttcaaatcccggaagaatatcttcagtactacacaccgaaaatcaacaacatctacaaactgcttgtcagaggcagccgcttatatccggatatttttctgtttcttggcttttgcaacgaatttcatgcgtatggctatgattttgaatacatcgcgcaaaaatggaaaagcaaaaaatacatcggatactggcagtctgaacatttctttcataaacatattctggatctgaaagaatttttcatcccgaaaaacgttagcgaacaagctaacttactggcagcgaaaatcttagaatctcagtcaagcctgtcaattcatatcagacgcggcgattacatcaaaaacaaaacagccacactgacacatggagtttgctctcttgaatactacaagaaagcactgaacaaaatcagagatttagccatgattcgcgatgtgtttatcttttctgatgatattttctggtgtaaagaaaacatcgaaacacttctgagcaaaaaatacaacatctactactcagaagatttaagccaagaagaagatttatggctgatgtctctggctaatcatcatattatcgccaactcttcattttcatggtggggcgcatatcttggaagctctgcgagccagattgttatctatccgacaccgtggtatgatattacaccgaaaaacacatacattcctatcgtgaaccattggatcaacgtggacaaacattcctcctgctaaaaaggaggaactactatgggcaatacatcaattcaaacacagagctatagagcagttgataaagatgcgggacaaagccgctcttatattatcccgtttgcactgctttgctctctgtttttcctgtgggctgtggccaataacctgaatgatattttactgccgcagtttcaacaggcgtttacacttacaaactttcaagctggattaattcagagcgccttttactttggctactttatcatcccgatcccggctggaatccttatgaaaaaactgtcttacaaagctggaattatcacaggcttatttctgtatgcactgggagcagctctgttttggcctgctgccgaaatcatgaattatacactgtttctggtcggactgtttattatcgcagcgggactgggctgtcttgaaacagctgccaacccgtttgtcacagttcttggaccggaatcaagcggccattttagactgaaccttgcacaaacatttaactcatttggagcaatcatcgcggttgtgtttggccagagcttaattctgtctaatgtcccgcatcaaagccaggatgttctggataaaatgtctccggaacaactttcagcgtataaacattcacttgtgttaagcgtccagacaccgtatatgattatcgttgcaatcgtgcttttagtcgcgctgcttattatgttaacaaaatttccggcactgcaatctgataaccattcagatgcgaaacagggctcatttagcgcttctttatcaagactggccagaattagacattggcgctgggctgtgttagcccaattttgctatgtcggagcacagacagcgtgttggtcatatctgatccgctatgctgtcgaagaaattccgggaatgacagccggctttgcagcgaattatcttacaggcacaatggtttgctttttcatcggaagatttacaggcacatggttaatttctcgctttgcaccgcataaagtgcttgctgcctatgcgttaatcgcaatggcgctgtgccttatttcagcttttgccggcggacatgtcggccttattgcattaacactgtgtagcgcgtttatgtctatccaatacccgacaatctttagcctgggaattaaaaaccttggccaggatacaaaatacggatcttcatttatcgttatgacaattatcggcggaggcattgttacaccggtgatgggatttgtgagcgatgcagcgggcaatatcccgacagctgaattaattccggctctgtgttttgccgttatctttatctttgctagatttcgctctcaaacagccacaaactaatctagagtcgacgtccaggcatcaaataaaacgaaaggctcagtcgaaagactgggcctttcgttttatctgttgtttgtcggtgaacgctctctactagagtcacactggctcaccttcgggtgggcctttctgcgtttatatacgcgttaacccgggcccgcggatggatatgatcagatcctttaactctggcaaccctcaaaattgaatgagacatgctacacctccggataataaatatatataaacgtatatagatttcataaagtctaacacactagacttatttacttcgtaattaagtcgttaaaccgtgtgctctacgaccaaaactataaaacctttaagaactttctttttttacaagaaaaaagaaattagataaatctctcatatcttttattcaataatcgcatccgattgcagtataaatttaacgatcactcatcatgttcatatttatcagagctcgtgctataattatactaattttataaggaggaaaaaatatgggcatttttagtatttttgtaatcagcacagttcattatcaaccaaacaaaaaataagtggttataatgaatcgttaataagcaaaattcatataaccaaattaaagagggttataatgaacgagaaaaatataaaacacagtcaaaactttattacttcaaaacataatatagataaaataatgacaaatataagattaaatgaacatgataatatctttgaaatcggctcaggaaaaggccattttacccttgaattagtaaagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatatggtaatataccttataacataagtacggatataatacgcaaaattgtttttgatagtatagctaatgagatttatttaatcgtggaatacgggtttgctaaaagattattaaatacaaaacgctcattggcattacttttaatggcagaagttgatatttctatattaagtatggttccaagagaatattttcatcctaaacctaaagtgaatagctcacttatcagattaagtagaaaaaaatcaagaatatcacacaaagataaacaaaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtgcctattttttgtgaatcgattcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

(19) United States
(12) Patent Application Publication
(10) Pub. No.: US 2022/0220524 A1
(43) Pub. Date: Jul. 14, 2022

(54) PRODUCTION OF FUCOSYLATED OLIGOSACCHARIDES IN BACILLUS

(71) Applicant: Chr Hansen HMO GmbH, Rheinbreitbach (DE)

(72) Inventors: Stefan JENNEWEIN, Bad Honnef (DE); Dirk WARTENBERG, Gau-Algesheim (DE); Stefanie HABERSETZER, Bad Honnef (DE)

(21) Appl. No.: 17/618,254

(22) PCT Filed: Jun. 8, 2020
(86) PCT No.: PCT/EP2020/065835

§ 371 Date: Dec. 10, 2021

(30) Foreign Application Priority Data
Jun. 12, 2019 (EP) 19179803.2

(51) Int. Cl. C12P 19/18; C12N 15/52; C12N 9/10; C12N 9/12; C12N 9/24

(52) U.S. Cl. CPC C12P 19/18; C12N 15/52; C12N 9/1051; C12Y 302/01023; C12N 9/1205; C12N 9/2402; C12Y 204/01069

(57) ABSTRACT

Disclosed are non-sporulating Bacillus cells for the production of a fucosylated oligosaccharide as well as a method for producing a fucosylated oligosaccharide, wherein said Bacillus cell has been genetically engineered to possess a lactose permease, a GDP-fucose biosynthesis pathway and a fucosyltransferase.

The present invention relates to the technical fields of genetic engineering, particularly to genetic engineering of Bacillus cells for the production of fucosylated oligosaccharides, and to the microbial production of fucosylated oligosaccharides using said genetically engineered Bacillus cells.

### BACKGROUND

Human breast milk has optimal nutritional properties for infants. The saccharides that are present in human breast milk represent the main component of human milk, ahead of fats and proteins. In addition to lactose, which serves as an energy source, human breast milk contains about 5 to 25 grams per liter of complex sugar molecules, i.e. oligosaccharides. These oligosaccharides are found at significant concentrations in only human milk, and they are collectively known as human milk oligosaccharides (HMOs).

Approximately 200 structurally distinct HMOs have been identified to date. Said HMOs are based on the disaccharide lactose (consisting of a glucose (Glc) moiety and a galactose (Gal) moiety) and bear additional monosaccharide residues which are based on N-acetyl-glucosamine (GlcNAc), fucose (Fuc), sialic acid (NeuNAc), and galactose (Gal). The concentration and composition of HMOs in human milk varies between individuals and during the lactation period from up to 20 g/L in the colostrum to 5-10 g/L in the mature milk.

Milk of women belonging to the so-called “secretor phenotype” contains a high content of α-1,2-fucosylated HMOs. These women express the FUT2 gene encoding the so-called “fucosyltransferase 2”. The most abundant HMOs in their milk are 2′-fucosyllactose (2′-FL; Fuc(α1-2)Gal(ρ1-4)Glc) and lacto-N-fucopentaose-I (LNPF-I; Fuc(α1-2)Gal(β1-3)GlcNAc(β1-3)Gal(β1-4)Glc).

Human milk oligosaccharides are not digested during their transit through the intestine of infants. Due to their persistence in the infant's gut, they exhibit beneficial effects. More specifically, HMOs have been shown to be prebiotic as they serve as carbon source for commensal microorganisms of the genera Bifidobacterium, Bacteroides and Lactobacillus. Therefore, HMOs support proliferation of these microorganisms in infants' guts.

Human milk oligosaccharides also directly reduce colonization of an infant's gut by pathogens in that they prevent adherence of said pathogens to glycan structures on the gut's mucosal surface. The HMOs function as a decoy due to their structural similarity to epithelial surface glycans and inhibit invasion of the pathogens thereby reducing the risk of infections.

Alpha-1,2-fucosylated HMOs have been shown to be protective against infections with Campylobacter jejuni, the causative agent of most common bacterial diarrheas. The α-1,2-fucosylated HMOs are also associated with protection against diarrhea caused by the heat stable toxin of Escherichia coli. Also, the risk of infections with diarrhea-mediating caliciviruses is reduced by a high content of α-1,2-fucosylated HMOs in breast milk. HMOs, especially the fucosylated HMO lacto-N-fucopentaose V (LNFP-V; Gal(β1-3)GlcNAc (β1-3)Gal(β1-4)[Fucα1-3]Glc), bind(s) to the carbohydrate binding site of toxin A from Clostridium difficile, the most common cause of nosocomial diarrhea. Thus, HMOs seem to prevent the interaction of toxin A from C. difficile with cellular receptors.

Furthermore, adherence of Pseudomonas aeruginosa to epithelial cells was significantly inhibited by 2′-FL and 3-fucosyllactose (3-FL; Gal(β1-4)[Fucα1-3]Glc). Binding of noroviruses (Norwalk-like viruses, NLV), the main cause of acute gastroenteritis, to histo-blood group antigens is prevented by α-1,2-fucosylated HMOs as well as by α-1,3-fucosylated HMOs. This indicates the potential of these HMOs to inhibit norovirus capsid-binding to host receptor glycans.

Due to the known benefits of HMOs, and especially of fucosylated HMOs, an economically worthwhile process for their synthesis is desired such that these oligosaccharides or at least some of these oligosaccharides become available as supplement for infant formula.

Due to the limited supply and difficulties of obtaining pure fractions of individual human milk oligosaccharides, chemical routes to the synthesis of some of these complex molecules were developed. However, neither chemical synthesis nor biocatalytic approaches proved to be commercially sustainable. Moreover, particularly chemical synthesis of human milk oligosaccharides involves the use of several noxious chemicals, which impose the risk to contaminate the final product.

Due to the challenges involved in the chemical synthesis of human milk oligosaccharides, fermentative approaches for producing HMOs were developed. Today, microbial production of several HMOs such as 2′-fucosyllactose, 3-fucosyllactose, lacto-N-tetraose, lacto-N-neotetraose, lacto-N-fucopentaose I, lacto-N-difucohexaose II, 3′-sialyllactose and 6′-sialyllactose using mainly genetically engineered Escherichia coli strains have been disclosed.

To date, recombinant E. coli cells are used for microbial production of HMOs in industrial scale. However, the genus Escherichia coli comprises pathogenic members as well as non-pathogenic members. Notwithstanding that non-pathogenic E. coli strains are employed for the microbial production of HMOs, such non-pathogenic E. coli are not recognized as safe for the manufacturing of products that are intended for human consumption in a variety of jurisdictions. Therefore, microbial cells of genera that are recognized as safe in such jurisdictions are needed for the manufacturing of compounds that are intended for human consumption to obtain regulatory approval of said products for their commercialization in said jurisdictions.

The problem is solved by using bacterial cells of the genus Bacillus which are recognized as being safe for human consumption, and which are capable of producing a fucosylated oligosaccharide.

Bacteria of the genus Bacillus are gram-positive, rod-shaped, endospore-forming microbial cells of either aerobic or facultatively anaerobic species. The genus Bacillus belongs to the phylum Firmicutes. The genome of the members of the genus Bacillus possesses a bias towards A-T base pairs in its codon usage. Bacillus species are almost ubiquitously present in nature. They can for example be found in soil (B. subtilis), but also occur in extreme environments such as high pH (B. alcalophilus), high temperature (B. thermophilus), or high salt (B. halodurans).

The genus Bacillus includes 266 named species which include free living species as well as parasitic pathogenic species. Two Bacillus species are considered of being medically significant: B. anthracis, which causes anthrax, and B. cereus, which causes food poisoning. A third species, B. thuringiensis, is an important insect pathogen producing a toxin that can kill insects. Thus, it is used as an insecticide to control insect pests.

Because of their GRAS (Generally Recognized as Safe) status, several Bacillus species, e.g. B. amyloliquefaciens, B. licheniformis and B. subtilis, are used in the biotechnological production of various proteins and compounds to be used in food and pharmaceutical industry.

B. amyloliquefaciens is the source of restriction enzyme BamHI, and also synthesizes the natural antibiotic protein barnase. In addition, B. amyloliquefaciens produces plantazolicin, an antibiotic with selective activity against B. anthracis. Alpha-amylase from B. amyloliquefaciens is often used in starch hydrolysis. B. amyloliquefaciens is also a source of subtilisin, which catalyzes the breakdown of proteins.

B. amyloliquefaciens is a root-colonizing bacterium that is used to fight some plant root pathogens in agriculture, aquaculture and hydroponics as it acts against bacterial and fungal pathogens, and may prevent infection by competitive exclusion or out-competing the unwanted pathogen.

Its high capacity of secreting alkaline serine protease has made B. licheniformis one of the most important bacteria in industrial enzyme production. Subtilisin Carlsberg secreted by B. licheniformis is used as a detergent protease, and is sold under the trade name Alcalase®.

B. subtilis is a catalase positive bacterium that is found in soil and the gastrointestinal tract of ruminants and humans. B. subtilis and substances derived from this endotoxin-free bacterium have been evaluated by different authoritative bodies for their safe and beneficial use in food. In the United States carbohydrase and protease enzymes from B. subtilis are recognized as generally safe (GRAS) by the US Food and Safety Administration (FDA). Bacillus subtilis has also been granted the “Qualified Presumption of Safety” status by the European Food Safety Authority.

Moreover, nontoxigenic and nonpathogenic strains of B. subtilis strain were commonly used in food. For example, fermented soy beans in the form of natto, which is commonly consumed in Japan, contains as many as 10⁸ viable B. subtilis cells per gram. The fermented beans are recognized for their contribution to a healthy gut flora and vitamin K₂ intake. The natto product and the B. subtilis var. natto as its principal component are FOSHU (Foods for Specified Health Use) approved by the Japanese Ministry of Health, Labor and Welfare as effective for preservation of health.

B. subtilis is easy to handle, grows rapidly and the culture conditions are simple. Recombinant B. subtilis strains are used in the production of polyhydroxyalkanolates, hyaluronic acid and various enzymes such as amylase and proteases.

Wild-type natural isolates of B. subtilis are difficult to work with as compared to laboratory strains that have undergone domestication processes of mutagenesis and selection. These domesticated strains often have improved capabilities to undergo natural competence development (uptake and integration of environmental DNA), growth, and loss of abilities needed “in the wild”. In B. subtilis, linear DNA as well as multimeric forms of plasmid DNA are actively taken up by natural competent cells.

Under certain physiological conditions, a small subpopulation of B. subtilis cells becomes competent. In B. subtilis, natural competence is regulated by a complex regulatory network. Key regulators in this network are, amongst others, the master regulator of competence ComK and the transcriptional master regulator of sporulation Spo0A. Transformation efficiency of B. subtilis cells and probably the efficacy for integrating DNA into their genome can be improved by genetic engineering. This can be achieved by ectopic integration of an expression cassette, comprising a regulated promoter (e.g. the mannitol-inducible P_{mtlA} promoter) and the genes comK and comS, into the genome of B. subtilis. Additionally, this strategy allows transformation of B. subtilis by natural competence using a complex medium (e.g. LB).

For the production of fucosylated saccharides, B. subtilis can be genetically modified by various methods.

Gene integration and/or (simultaneous) gene inactivation by disruption or deletion can be achieved by homologous recombination. For an efficient homologous recombination, at least 400-500 bp of homology arms are necessary in B. subtilis.

Another method for targeted genome engineering is the modern CRISPR-Cas9 system. This rapid and markerless genome-editing tool can be used for large scale genomic deletions, small and large DNA insertions, gene silencing by introduction of a stop codon as well as introduction of point mutations. No previous genome modifications are required for the scarless genome editing by CRISPR-Cas9.

Random chromosomal integration of genes and insertional mutagenesis can be performed using a modified mariner-derived transposon. This system is not biased toward hotpots in B. subtilis while showing high efficiency in random ectopic integration.

Although Bacillus species are used for industrial scale production of enzymes, Bacillus cells have not been implemented to date for industrial scale production of oligosaccharides, in particular of fucosylated oligosaccharides.

Chinese patent application CN 108 410 787 A discloses recombinant Bacillus subtilis cells which synthesizes lactyl-N-neotetraose. Said recombinant B. subtilis cells possess a lactose permease gene which has been integrated into the cell's genome. In addition, said Bacillus cell which bears a plasmid comprising a beta-1,3-N-glucosamine transferase gene and a β-1,4-galactosyltransferase gene. The B. subtilis cells may be cultivated in the presence of exogenous lactose, and synthesizes lactyl-N-neotetraose at titers of up to about 1 g/L which is too little for economically reasonable industrial scale production.

Although a variety of patent applications mention Bacillus as a genus which is suitable for the production of oligosaccharides such as lacto-N-neotetraose, no commercial use of Bacillus for the productions of fucosylated oligosaccharides, in particular of fucosylated human milk oligosaccharides has been implemented yet, presumably because of the significant efforts in metabolic engineering that are required to implement the necessary biosynthetic pathways for HMO production in Bacillus. Whereas above-referenced B. subtilis for the production of LNnT relies on donor substrates that are naturally occurring in B. subtilis cells, production of a fucosylated oligosaccharide in Bacillus requires implementation of a heterologous metabolic pathway in the cell for providing the required donor substrate GDP-fucose.

Chinese patent application CN 109 735 479 A discloses recombinant Bacillus subtilis cells for synthesizing 2′-fucosyllactose, wherein the expression of a lactose transport enzyme is enhanced, and which cell expresses a fucose kinase, a phosphate guanine transferase and a fucosyltransferase. The yield of 2′-fucosyllactose in the fermentation medium was reported to be between 0.424 g/L and 1.042 g/L.

It was nonetheless an object of the present invention to provide a bacterial cell of the genus Bacillus for the production of fucosylated oligosaccharides.

The object has been achieved by providing a Bacillus cell that possesses a lactose permease for import of exogenous lactose into the cell, a de novo GDP-fucose biosynthesis pathway for providing GDP-fucose, and a fucosyltransferase for transfer of a fucose moiety from GDP-fucose to lactose. Said Bacillus cell can be cultivated in the presence of exogenous lactose to produce a fucosylated oligosaccharide.

### SUMMARY

According to a first aspect, provided is a non-sporulating bacterial cell of the genus Bacillus for the production of a fucosylated oligosaccharide, wherein the Bacillus cell possesses a lactose permease, a GDP-fucose biosynthesis pathway, and a fucosyltransferase.

According to a second aspect, provided is the use of the non-sporulating bacterial cell of the genus Bacillus according to the first aspect for the production of a fucosylated oligosaccharide.

According to a third aspect, provided is a method for the production of a fucosylated oligosaccharide, the method comprising:
providing a non-sporulating bacterial cell of the genus Bacillus, wherein said Bacillus cell possesses a lactose permease, a GDP-fucose biosynthetic pathway, and a lactose-accepting fucosyltransferase;
cultivating the Bacillus cell in a culture medium containing lactose and under conditions that are permissive for the Bacillus cell to produce the fucosylated oligosaccharide, and
optionally retrieving the fucosylated oligosaccharide from the culture medium and/or the Bacillus cell.

According to a fourth aspect, provided are fucosylated oligosaccharides that were produced by a Bacillus cell which possesses a lactose permease, a GDP-fucose biosynthesis pathway and a fucosyltransferase.

According to a fifth aspect, provided is the use of a fucosylated oligosaccharide that was produced by a Bacillus cell which possesses a lactose permease, a GDP-fucose biosynthesis pathway and a fucosyltransferase for the manufacturing of a nutritional composition.

According to a sixth aspect, provided are nutritional compositions containing at least one fucosylated oligosaccharide that had been produced by a Bacillus cell which possesses a lactose permease, a GDP-fucose biosynthesis pathway and a fucosyltransferase.

### DETAILED DESCRIPTION

The present invention concerns Bacillus cells for the production of fucosylated oligosaccharides, their use and methods for producing fucosylated oligosaccharides by cultivating said Bacillus cells in a culture medium containing lactose, and under conditions that are permissive for the production of said fucosylated oligosaccharide by said Bacillus cell.

For being able to produce a fucosylated oligosaccharide, a Bacillus cell has to provide a donor substrate comprising a fucose moiety, and an acceptor substrate being a disaccharide or an oligosaccharide to a fucosyltransferase such that the fucosyltransferase can transfer the fucose moiety from the donor substrate to said acceptor substrate thereby generating the fucosylated oligosaccharide.

It is to be understood that the fucosylated oligosaccharide, said Bacillus cell is intended to produce, is the desired fucosylated oligosaccharide, whereas other fucosylated oligosaccharides that may be generated due to the promiscuity of the fucosyltransferase during production of the desired fucosylated oligosaccharide are considered as undesired fucosylated oligosaccharides or by-products. A suitable donor substrate is GDP-L-fucose, and a suitable acceptor substrate for generating a fucosyllactose is the disaccharide lactose. The resulting desired fucosylated oligosaccharide is 2′-fucosyllactose, 3-fucosyllactose or 2′,3-difucosyllactose.

Wild-type Bacillus cells neither synthesize lactose intracellularly nor do they internalize and metabolize exogenous lactose. However, lactose is the acceptor substrate for a fucose moiety by a lactose-accepting fucosyltransferase in the formation of some fucosylated oligosaccharides. Hence, to be able to produce a fucosylated oligosaccharide such as 2′-fucosyllactose, 3-fucosyllactose or 2′,3-difucosyllactose, a Bacillus cell has to have the capability of providing lactose to the lactose-accepting fucosyltransferase, either by generating lactose intracellularly and/or by internalizing exogenous lactose.

In an embodiment, the Bacillus cell for producing a fucosylated oligosaccharide is able to internalize exogenous lactose by possessing a lactose permease. The term “exogenous” with respect to lactose as used herein refers to lactose that does not originate from the Bacillus cell, i.e. being intracellularly synthesized by the Bacillus cell, but which originates from outside the Bacillus cell and is added to the culture medium in which the Bacillus cell is grown to produce the fucosylated oligosaccharide.

In an additional and/or alternative embodiment, the Bacillus cell has been genetically engineered to be able to internalize exogenous lactose, i.e. to possess a lactose permease. Thus, the Bacillus cell for producing a fucosylated oligosaccharide possesses a heterologous lactose permease. A suitable lactose permease is E. coli LacY or a functional variant thereof.

The term “heterologous” as used herein with respect to proteins, polypeptides, enzymes and transporters, but also with respect to nucleic acid molecules and/or nucleotide sequences, refers to a molecule that is non-native to the species of cell which contains said molecule. The term “non-native” indicates that said molecule is not present in the naturally occurring or wild-type progenitor cell, i.e. the cell of the same species that is most commonly occurring in nature, of the Bacillus cell.

The term “functional variant” as used herein with respect to enzymes and/or transport molecules refers to proteins or polypeptides possessing the same activity (enzymatic, catalytic or translocating) as the referenced enzyme or transporter, but which has a different amino acid sequence than the referenced enzyme or transporter molecule. Thus, a typical variant of a protein/polypeptide differs in amino acid sequence from the reference protein/polypeptide. A variant and the reference protein/polypeptide may differ in amino acid sequence by one or more substitutions, additions, and/or deletions in any combination. Hence, the term “functional variant” comprises truncated versions of the referenced protein/polypeptide which possesses the same activity as the reference protein/polypeptide. A substituted or inserted amino acid residue may or may not be one encoded by the genetic code. A variant of a protein/polypeptide may be a naturally occurring such as an allelic variant, or it may be a variant that is not known to occur naturally. Non-naturally occurring variants of proteins/polypeptides may be made by mutagenesis techniques, by direct synthesis, and by other recombinant methods known to the persons skilled in the art. Within the scope of the present disclosure, also proteins and interspecies homologs are comprised by the term “variant”, that have an amino acid sequence/nucleic acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to the referenced polypeptide.

The term “same activity” as used herein refers to an enzymatic, catalytic or transporting activity of a protein/polypeptide in qualitative manner only. Hence, “functional variant” also comprises variants which possess an increased or decreased activity as compared to the activity of the referenced protein/polypeptide.

In an additional and/or alternative embodiment, the Bacillus cell has been genetically engineered to contain and express a nucleotide sequence which encodes a lactose permease, preferably a nucleotide sequence that encodes the E. coli lactose permease LacY or a functional variant thereof.

The E. coli lactose permease LacY is encoded by the protein coding region (i.e. the open reading frame) of the E. coli lacY gene (Gen Bank accession number: NP_414877.1). Hence, in an embodiment, the Bacillus cell has been genetically engineered to contain and express a nucleotide sequence that encodes the E. coli lactose permease LacY or a functional variant thereof.

In an additional and/or alternative embodiment, the codon usage of the nucleotide sequence encoding the heterologous lactose permease is adapted to the codon usage of Bacillus. The codon usage of B. subtilis for example is peculiar in that the overall GC-content is below about 45%, the GC-content of the first letter of the codons above about 51%, the CG-content of the second letter of the codons below about 36.1%, and the CG-content of the third letter of the codons below about 46%.

For expression of the lactose permease, the Bacillus cell contains a recombinant lactose permease gene, wherein the nucleotide sequence which encodes the lactose permease is operably linked to expression control sequences which mediate expression of the lactose permease open reading frame.

The term “operably linked” as used herein, shall mean a functional linkage between a nucleic acid/gene expression control sequence (such as a promoter, signal sequence, or array of transcription factor binding sites) and a second nucleic acid sequence (typically referred to as “protein coding region”, “open reading frame”, and sometimes even as “gene”), wherein the expression control sequence effects transcription and/or translation of the nucleotide sequence corresponding to the second sequence. Accordingly, the term “promoter” designates DNA sequences which usually “precede” an open reading frame in a DNA polymer and provide a site for initiation of the transcription into mRNA. “Regulator” DNA sequences, also usually “upstream” of (i.e., preceding) an open reading frame in a given DNA polymer, bind proteins that determine the frequency (or rate) of transcriptional initiation. Collectively referred to as “promoter/regulator” or “control” DNA sequence, these sequences which precede a selected open reading frame (or series of open reading frames) in a functional DNA polymer cooperate to determine whether the transcription (and eventual expression) of an open reading frame will occur. DNA sequences which “follow” a gene in a DNA polymer and provide a signal for termination of the transcription into mRNA are referred to as transcription “terminator” sequences.

The recombinant lactose permease gene may be integrated into the Bacillus chromosome, or present as an episomal version on a plasmid within the Bacillus cell.

Expression of the heterologous lactose permease gene in the Bacillus cell enables the resulting Bacillus cell to internalize exogenously supplied lactose from the culture medium. The internalized lactose can then serve as an acceptor substrate for a fucosyltransferase (see herein below).

The Bacillus cell for producing a fucosylated oligosaccharide has to be able to provide a donor substrate for transferring a fucose moiety to an acceptor substrate. The donor substrate for a fucose moiety is GDP-fucose. Hence, the Bacillus cell has to be able to intracellularly produce GDP-fucose. For intracellular biosynthesis of GDP-fucose, the Bacillus cell possesses a GDP-fucose biosynthesis pathway. The Bacillus has been genetically engineered to possess a GDP-fucose biosynthesis pathway. The GDP-fucose biosynthesis pathway may either be a de novo GDP-fucose biosynthesis pathway and/or a GDP-fucose salvage pathway.

In an embodiment, the Bacillus cell possesses a de novo GDP-fucose biosynthesis pathway for intracellular biosynthesis of GDP-fucose. The Bacillus cell has been genetically engineered to possess the de novo GDP-fucose biosynthesis pathway.

The de novo GDP-fucose biosynthesis pathway comprises activities of the following enzymes:
1. a fructose-6-phosphate isomerase;
2. a phosphomannomutase;
3. a GDP:mannose-1-phosphate guanylyltransferase;
4. a GDP-mannose-4,6-dehydratase; and
5. a GDP-fucose synthase.

Hence, the Bacillus cell for producing a fucosylated oligosaccharide possesses a fructose-6-phosphate isomerase (typically also designated as mannose-6-phosphate isomerase), a phosphomannomutase, a GDP:mannose-1-phosphate guanylyltransferase, a GDP-mannose-4,6-dehydratase, and a GDP-fucose synthase.

In an additional and/or alternative embodiment, the Bacillus cell has been genetically engineered to possess a fructose-6-phosphate isomerase, a phosphomannomutase, a GDP:mannose-1-phosphate guanylyltransferase, a GDP-mannose-4,6-dehydratase, and a GDP-fucose synthase.

The de novo pathway of GDP-fucose biosynthesis starts from isomerization of fructose-6-phosphate to mannose-6-phosphate, a reaction that is catalyzed by the fructose-6-phosphate isomerase. In an additional and/or alternative embodiment, the Bacillus cell has been genetically engineered to contain and express a nucleotide sequence which encodes a fructose-6-phosphate isomerase. A suitable fructose-6-phosphate is E. coli ManA or a functional variant thereof. An exemplary nucleotide sequence is the nucleotide sequence which encodes E. coli ManA.

The E. coli fructose-6-phosphate isomerase ManA is encoded by the protein coding region of the E. coli manA gene (GenBank accession number: NP_416130.3). Thus, in an additional embodiment, the Bacillus cell has been genetically engineered to contain and express a nucleotide sequence which encodes the E. coli ManA or a functional variant thereof.

The E. coli fructose-6-phosphate isomerase ManA is encoded by the open reading frame of the E. coli manA gene (Gen Bank accession number: NP_416130.3). Thus, in an additional and/or alternative embodiment, the Bacillus cell contains the E. coli manA gene or a functional variant thereof.

In an additional and/or alternative embodiment, the codon usage of the nucleotide sequence encoding the fructose-6-phosphate isomerase is adapted to the codon usage of Bacillus.

Other suitable fructose-6-phosphate isomerases are B. subtilis ManA and the paralogous proteins B. subtilis GmuF (YdhS) and B. subtilis Pmi (YvyI) or functional variants thereof. Exemplary nucleotide sequences are the nucleotide sequences which encodes B. subtilis ManA, B. subtilis GmuF or B. subtilis Pmi. The B. subtilis fructose-6-phosphate isomerase ManA is encoded by the open reading frame of the B. subtilis manA gene (Gen Bank accession number: NP_389084.1).

The B. subtilis fructose-6-phosphate isomerase ManA is encoded by the protein coding region of the B. subtilis manA gene (GenBank accession number: (NP_389084.1). Thus, in an additional embodiment, the Bacillus cell has been genetically engineered to contain and express a nucleotide sequence which encodes the B. subtilis ManA or a functional variant thereof. Thus, in an additional and/or alternative embodiment, the Bacillus cell contains the B. subtilis manA gene or a functional variant thereof. In another additional and/or alternative embodiment, the Bacillus cell has been genetically engineered to contain and express a nucleotide sequence which encoded the B. subtilis gmuF gene or a functional variant thereof and/or a nucleotide sequence which encodes the B. subtilis pmi gene or a functional variant thereof.

In an additional and/or alternative embodiment, the expression of the native manA gene, the native gmuF gene and/or the native pmi gene can be increased. The expression of the native manA gene, the native gmuF gene and/or the native pmi gene can be increased in that the endogenous promoter of at least one of the native manA gene, the native gmuF gene and the native pmi gene is replaced by a stronger promoter, i.e. a promoter mediating an increased expression as compared to the native promoter of the manA gene, the gmuF gene and the pmi gene respectively. In another additional or alternative embodiment, the expression of the native manA gene, the native gmuF gene and/or the native pmi gene can be enhanced in that additional copies of the native manA gene, the native gmuF gene and/or the native pmi gene are propagated in the Bacillus cell.

In another additional or alternative embodiment, the expression of the native manA gene can be enhanced by deletion or functional inactivation of the manP gene encoding a mannose transporter leading to a constitutive expression of the native manA gene.

In another additional or alternative embodiment, the expression of the native gmuF gene can be enhanced by deletion or functional inactivation of the gmuR gene encoding the corresponding transcriptional repressor.

For expression of the fructose-6-phosphate isomerase, the Bacillus cell contains a recombinant fructose-6-phosphate isomerase gene, wherein the nucleotide sequence which encodes the fructose-6-phosphate isomerase is operably linked to expression control sequences.

The recombinant fructose-6-phosphate isomerase gene may be integrated into the Bacillus chromosome or present as an episomal version on a plasmid within the Bacillus cell.

In the second step of the de novo GDP-fucose biosynthesis, mannose-6-phosphate is converted to mannose-1-phosphate by the enzymatic activity of a phosphomannomutase. In an additional and/or alternative embodiment, the Bacillus cell has been genetically engineered to contain and express a nucleotide sequence which encodes a phosphomannomutase.

A suitable phosphomannomutase is the E. coli phosphomannomutase ManB. Thus, in an additional embodiment, the Bacillus cell has been genetically engineered to contain and express a nucleotide sequence which encodes E. coli ManB or a functional variant thereof.

The E. coli phosphomannomutase ManB is encoded by the nucleotide sequence of the protein coding region of the E. coli manB gene (Gen Bank accession number: NP_416552.1). Thus, in an additional and/or alternative embodiment, the Bacillus cell contains the E. coli manB gene or a functional variant thereof.

In an additional and/or alternative embodiment, the codon usage of the nucleotide sequence encoding the phosphomannomutase is adapted to the codon usage of Bacillus.

For expression of the phosphomannomutase, the Bacillus cell contains a recombinant phosphomannomutase gene, wherein the nucleotide sequence which encodes the phosphomannomutase is operably linked to expression control sequences.

The recombinant phosphomannomutase gene may be integrated into the Bacillus chromosome or present in the Bacillus cell as an episomal version on a plasmid.

The next step in the de novo biosynthesis pathway of GDP-fucose is the formation of GDP-mannose by the following reaction:

α-D-mannose-1-phosphate+GTP+H⁺=>diphosphate+GDP-α-D-mannose

The conversion of mannose-1-phosphate to GDP-mannose is mediated by a GDP:mannose-1-phosphate guanylyltransferase. Thus, the Bacillus cell for producing a fucosylated oligosaccharide possesses a heterologous GDP:mannose-1-phosphate guanylyltransferase. A suitable GDP:mannose-1-phosphate guanylyltransferase is E. coli ManC or a functional variant thereof.

In an additional and/or alternative, the Bacillus cell has been genetically engineered to contain and express a nucleotide sequence which encodes a GDP:mannose-1-phosphate guanylyltransferase. An exemplary nucleotide sequence is the nucleotide sequence which encodes E. coli ManC. Thus, in an additional embodiment, the Bacillus cell has been genetically engineered to contain and express a nucleotide sequence which encodes the E. coli ManC or a functional variant thereof.

The E. coli GDP:mannose-1-phosphate guanylyltransferase ManC is encoded by the open reading frame of the E. coli manC gene (Gen Bank accession number: NP_416553.1). Thus, in an additional and/or alternative embodiment, the Bacillus cell contains the E. coli manC gene or a functional variant thereof.

In an additional and/or alternative embodiment, the codon usage of the nucleotide sequence encoding the GDP:mannose-1-phosphate guanylyltransferase is adapted to the codon usage of Bacillus.

For expression of the GDP:mannose-1-phosphate guanylyltransferase, the Bacillus cell contains a recombinant GDP:mannose-1-phosphate guanylyltransferase gene, wherein the nucleotide sequence which encodes the GDP:mannose-1-phosphate guanylyltransferase is operably linked to expression control sequences.

The recombinant GDP:mannose-1-phosphate guanylyltransferase gene may be integrated into the Bacillus chromosome or present as an episomal version on a plasmid.

In the subsequent step of the de novo GDP-fucose pathway, catalyzed by a GDP-mannose-4,6-dehydratase, GDP-D-mannose is converted to GDP-4-dehydro-6-deoxy-D-mannose.

Thus, the Bacillus cell for producing a fucosylated oligosaccharide possesses a GDP-mannose-4,6-dehydratase. A suitable GDP-mannose-4,6-dehydratase is E. coli Gmd.

In an additional and/or alternative, the Bacillus cell has been genetically engineered to contain and express a nucleotide sequence which encodes a GDP-mannose-4,6-dehydratase. An exemplary nucleotide sequence is the nucleotide sequence which encodes E. coli Gmd. Thus, in an additional embodiment, the Bacillus cell has been genetically engineered to contain and express a nucleotide sequence which encodes the E. coli Gmd or a functional variant thereof.

The E. coli GDP-mannose-4,6-dehydratase Gmd is encoded by the open reading frame of the E. coli gmd gene (Gen Bank accession number: NP_416557.1). Thus, in an additional and/or alternative embodiment, the Bacillus cell contains the E. coli gmd gene or a functional variant thereof.

In an additional and/or alternative embodiment, the codon usage of the nucleotide sequence encoding the GDP-mannose-4,6-dehydratase is adapted to the codon usage of Bacillus.

For expression of the GDP-mannose-4,6-dehydratase, the Bacillus cell contains a recombinant GDP-mannose-4,6-dehydratase gene, wherein the nucleotide sequence which encodes the GDP-mannose-4,6-dehydratase is operably linked to expression control sequences.

The recombinant GDP-mannose-4,6-dehydratase gene may be integrated into the Bacillus chromosome or present as an episomal version on a plasmid.

In the final reaction of the de novo GDP-fucose biosynthesis pathway, GDP-4-dehydro-6-deoxy-D-mannose is converted to GDP-fucose by a two-step NADPH-dependent reaction. This conversion is mediated by the GDP-fucose synthase and involves an epimerase reaction and a reductase reaction. Said epimerase reaction converts GDP-4-keto-6-deoxymannose to GDP-4-keto-6-deoxygalactose, which is then reduced to GDP-fucose.

Thus, the Bacillus cell for producing a fucosylated oligosaccharide possesses a GDP-fucose synthase. A suitable GDP-fucose synthase is E. coli WcaG or a functional variant thereof.

In an additional and/or alternative, the Bacillus cell has been genetically engineered to contain and express a nucleotide sequence which encodes a GDP-fucose synthase. An exemplary nucleotide sequence is the nucleotide sequence which encodes E. coli WcaG. Thus, in an additional embodiment, the Bacillus cell has been genetically engineered to contain and express a nucleotide sequence which encodes the E. coli WcaG or a functional variant thereof.

The E. coli GDP-fucose synthase WcaG is encoded by the open reading frame of the E. coli wcaG gene (Gen Bank accession number: NP_416556.1). Thus, in an additional and/or alternative embodiment, the Bacillus cell contains the E. coli wcaG gene or a functional variant thereof.

In an additional and/or alternative embodiment, the codon usage of the nucleotide sequence encoding the GDP-fucose synthase is adapted to the codon usage of Bacillus.

For expression of the GDP-fucose synthase, the Bacillus cell contains a recombinant GDP-fucose synthase gene, wherein the nucleotide sequence which encodes the GDP-fucose synthase is operably linked to expression control sequences.

The recombinant GDP-fucose synthase gene may be integrated into the Bacillus chromosome or present as an episomal version on a plasmid.

In an additional and/or alternative embodiment, the Bacillus cell possesses a GDP-fucose salvage pathway for the intracellular biosynthesis of GDP-fucose. In the salvage pathway of GDP-L-fucose, free cytosolic fucose is phosphorylated by L-fucokinase to form L-fucose-L-phosphate, which is then further converted to GDP-L-fucose.

The salvage pathway for biosynthesis of GDP-fucose comprises the following enzymes:

I. a fucose kinase; and

II. a L-fucose-1-phosphate guanylyltransferase.

Hence, the Bacillus cell for producing a fucosylated oligosaccharide which possesses a GDP-fucose salvage pathway possesses a fucose kinase and a L-fucose-1-phosphate guanylyltransferase.

In an additional and/or alternative embodiment, the Bacillus cell has been genetically engineered to possess a fucose kinase and a L-fucose-1-phosphate guanylyltransferase.

The fucose kinase, also called fucokinase, ATP:6-deoxy-L-galactose 1-phosphotransferase, ATP:β-L-fucose 1-phosphotransferase or L-fucokinase activity, L-fucose kinase activity catalyzes the reaction L-fucose+ATP→β-L-fucose-1-phosphate+ADP+2H⁺.

The L-fucose-1-phosphate guanylyltransferase or GDP-L-fucose pyrophosphorylase then converts said β-L-fucose-1-phosphate to GDP-L-fucose.

In an additional and/or alternative embodiment, the Bacillus cell has been genetically engineered to contain and express a nucleotide sequence which encodes a fucose kinase or a functional variant thereof, and a L-fucose-1-phosphate guanylyltransferase or a functional variant thereof.

In an additional and/or alternative embodiment, the fucose kinase and the L-fucose-1-phosphate guanylyltransferase are combined in a single polypeptide. Suitable genes for transformation, coding for a fucose kinase, a fucose-1-phosphate guanylyltransferase and/or a bifunctional L-fucose-1-phosphate guanylyltransferase can be obtained from the genera Bacteroides, Lentisphaera, Ruminococcus, Solibacter, Arabidopsis, Oryza, Physcomitrella, Vitis, Danio, Bos, Equus, Macaca, Pan, Homo, Rattus, Mus and Xenopus. An example for a bifunctional fucose kinase/L-fucose-1-phosphate guanylyltransferase is found in Bacteroides fragilis.

In B. fragilis, the bifunctional fucose kinase/L-fucose-1-phosphate guanylyltransferase Fkp is encoded by the B. fragilis fkp gene (GenBank accession number AY849806).

In an additional and/or alternative embodiment, the Bacillus cell has been genetically engineered to contain and express a nucleotide sequence that encodes the B. fragilis Fkp or a functional variant thereof.

In an additional and/or alternative embodiment, the codon usage of the nucleotide sequences encoding the fucose kinase, fucose-1-phosphateguanylyltransferase and/or the bifunctional fucose kinase/L-fucose-1-phosphate guanylyltransferase is adapted to the codon usage of Bacillus.

For expression of the fucose kinase and the fucose-1-phosphateguanylyltransferase, the Bacillus cell contains at least one recombinant gene, wherein the protein-coding region encoding the fucose kinase and the L-fucose-1-phosphate guanylyltransferase and/or the bifunctional fucose kinase/L-fucose-1-phosphate guanylyltransferase fucose kinase is operably linked to expression control sequences.

The recombinant fucose kinase gene, fucose-1-phosphateguanylyltransferase gene and/or the bifunctional fucose kinase/L-fucose-1-phosphate guanylyltransferase gene may be integrated into the Bacillus chromosome or present as an episomal version on a plasmid.

For the intracellular formation of fucosyllactose, the Bacillus cell possesses a fucosyltransferase. The enzymatic activity of the fucosyltransferase transfers the fucose moiety from the donor substrate to the acceptor substrate. For the biosynthesis of a fucosyllactose, said acceptor substrate is lactose. Hence, the fucosyltransferase is a lactose-accepting fucosyltransferase.

The fucosyltransferase is selected from the group consisting of α-1,2-fucosyltransferases for the biosynthesis of 2′-fucosyllactose, and α-1,3-fucosyltransferases for the biosynthesis of 3-fucosyllactose.

In an additional and/or alternative, the Bacillus cell has been genetically engineered to contain and express at least one nucleotide sequence which encodes a fucosyltransferase.

For producing 2′-fucosyllactose (2′-FL), the α-1,2-fucosyltransferases WbgL from E. coli O126 and FucT2 from Helicobacter pylori (EP 2 479 263 B1), the α-1,2-fucosyltransferases WblA from Vibrio cholera 022, FutD from H. bilis ATCC 437879, FutE from H. cinaede CCUG 18818, FutN from Bacteroides vulgatus ATCC 8482, FutO from Bacteroides ovatus ATCC 8483, WbgN from E. coli O55:H7, Bft1 and Bft3 from Bacteroides fragilis NCTC 9343 (WO 2014/018596 A2), and the α-1,2-fucosyltransferases FucT2 from H. pylori for the synthesis of Lewis Y and Lewis B saccharides (U.S. Pat. No. 6,670,160 B2) were described and are suitable α-1,2-fucosyltransferases for 2′-FL biosynthesis in Bacillus cells.

For producing 3-fucosyllactose, the α-1,3-fucosyltransferase Amuc from Akkermansia muciniphila, and FucT6 and FucT7 from Bacteroides fragilis (EP 2 439 264 A1), the α-1,3-fucosyltransferase FutA from H. pylori (US 2014/0120611 A1) are described and are suitable α-1,3-fucosyltransferases for 3-FL biosynthesis in Bacillus cells. In addition, WO 2016/040531 A1 discloses the α-1,3-fucosyltransferase CafC from B. nordii CL02T12C05 for the synthesis of 3-fucosyllactose and lactodifucotetraose, and CafD from H. hepaticus ATCC51449 for the production of LNnFP-III.

Additional fucosyltransferases that can be expressed in Bacillus cells for the production of fucosylated saccharides are disclosed in WO 2019/0088133 A1, which is incorporated herein by reference.

In an additional and/or alternative embodiment, the codon usage of the nucleotide sequence encoding the fucosyltransferase is adapted to the codon usage of Bacillus.

For expression of the fucosyltransferase, the Bacillus cell contains at least one recombinant gene, wherein the protein-coding region encoding the fucosyltransferase is operably linked to expression control sequences.

The recombinant fucosyltransferase gene may be integrated into the Bacillus chromosome or present as an episomal version on a plasmid.

In an additional and/or alternative embodiment, the Bacillus cell for producing a fucosylated oligosaccharide is able to internalize L-fucose. The capability of internalizing L-fucose is of advantage for Bacillus cells which possess the GDP-fucose salvage pathway.

In an additional and/or alternative embodiment, the Bacillus cell has been genetically engineered to enable or improve internalization of L-fucose. Thus, the Bacillus cell possesses a heterologous L-fucose permease. A suitable L-fucose permease is E. coli FucP or a functional variant thereof.

In an additional and/or alternative embodiment, the Bacillus cell as been genetically engineered to contain and express a nucleotide sequence which encodes a L-fucose permease, preferably a nucleotide sequence that encodes the E. coli FucP or a functional variant thereof.

The E. coli L-fucose permease is encoded by the protein coding region of the E. coli fucP gene (Gen Bank accession number: NP_417281.1). Hence, the Bacillus cell has been genetically engineered to contain and express a nucleotide sequence that encodes the E. coli FucP or a functional variant thereof.

In an additional and/or alternative embodiment, the codon usage of the nucleotide sequence encoding the L-fucose permease is adapted to the codon usage of Bacillus.

For expression of the L-fucose permease, the Bacillus cell contains a recombinant L-fucose permease gene, wherein the nucleotide sequence which encodes the L-fucose permease is operably linked to expression control sequences.

The recombinant L-fucose permease gene may be integrated into the Bacillus chromosome or present as an episomal version on a plasmid.

In an additional and/or alternative embodiment, the Bacillus cell for producing a fucosylated oligosaccharide lacks any β-galactosidase activity or possesses reduced β-galactosidase activity as compared to a wild type Bacillus cell of the same species.

The intracellular biosynthesis of fucosylated oligosaccharides requires import of lactose as acceptor substrate for the lactose-accepting fucosyltransferase. Any intracellular enzyme activity that hydrolyzes internalized lactose would affect the efficacy of fucosyllactose formation as the pool of intracellular lactose would be diminished. Therefore, it would be advantageous if the Bacillus cell for producing a fucosylated oligosaccharide would lack or at least possess—as compared to the wild-type Bacillus cell—reduced beta-galactosidase activity.

In an additional and/or alternative embodiment, the Bacillus cell has been genetically engineered to abolish or at least reduce the cell's β-galactosidase activity.

In an additional and/or alternative embodiment, the Bacillus cell has been genetically engineered by deletion or functional inactivation of the ganA gene. In another embodiment, the Bacillus cell has been genetically engineered to reduce the expression level of the ganA gene—as compared to the wild-type Bacillus cell.

The Bacillus ganA gene is also called yvfN or lacA. It is a gene of the GanR regulon which contains genes encoding enzymes involved in the utilization of galactan. The ganA gene encodes a beta-galactosidase that is involved in the galactan utilization of Bacillus.

Deletion or functional inactivation of the ganA gene abolishes the GanA-mediated β-galactosidase activity in the Bacillus cell, whereas reduction of the ganA expression lowers the amount of GanA in the Bacillus cell and hence the β-galactosidase activity which could interfere with the biosynthesis of a fucosylated oligosaccharide.

In an additional and/or alternative embodiment, the Bacillus cell has been genetically engineered by deletion or functional inactivation of the yesZ gene. The Bacillus yesZ gene encodes the beta-galactosidase YesZ which plays a role in the degradation of rhamnogalacturonan being derived from plant cell walls. The expression of Bacillus yesZ gene is induced by rhamnogalacturonan I. In another embodiment, the Bacillus cell has been genetically engineered to reduce the expression level of the yesZ gene—as compared to the wild-type Bacillus cell.

Deletion or functional inactivation of the yesZ gene abolishes the YesZ-mediated β-galactosidase activity in the Bacillus cell, whereas reduction of the yesZ expression lowers the amount of YesZ in the Bacillus cell and hence the β-galactosidase activity which could interfere with the biosynthesis of a fucosylated oligosaccharide.

When B. subtilis enters the post-exponential growth phase, they (start to) produce large quantities of extracellular proteases. Foreign proteins are often protease sensitive. Therefore, an exoprotease-free strain is desirable to increase the stability of heterologous proteins and to allow accumulation of high levels of foreign proteins. The genome of Bacillus encodes for at least eight extracellular proteases, namely nprE, aprE, epr, bpr, mpr, nprB, vpr and wprA. Thus, in an additional and/or alternative embodiment, the Bacillus cell has been genetically engineered in that at least one gene encoding for an extracellular protease has been deleted or functionally inactivated, preferably at least one of the genes selected from the group consisting of nprE, aprE, epr, bpr, mpr, nprB, vpr and wprA. Preferably, two, three, four, five six, seven or eight of the genes selected from the group consisting of nprE, aprE, epr, bpr, mpr, nprB, vpr and wprA have been deleted or functionally inactivated.

B. subtilis synthesizes pulcherriminic acid when growing in media containing a carbohydrate such as glucose or lactose. Excreted pulcherriminic acid forms the red pigment pulcherrimin, the salt of pulcherriminic acid (a ferric chelate), when iron is present in the growth medium. The formation of this undesired by-product during fermentation processes can be avoided/abolished by deletion or disruption of the genes yvmC and/or cypX. The gene yvmC encodes a cyclodipeptide synthase and the gene cypX encodes a cytochrome P450 cyclo-l-leucyl-l-leucyl dipeptide oxidase.

Thus, in an additional and/or alternative embodiment, the Bacillus cell has been genetically engineered in that at least one of the genes yvmC and cypX has been deleted or functionally inactivated.

The rhizobacterium B. subtilis possesses genes for the synthesis of more than 20 antibiotics. Among them are peptide antibiotics like Bacillus subtilis lantibiotics and lantibiotic-like peptides (subtilin, ericin S, mersacidin, sublancin 168, subtilosin A) and non-ribosomally synthesized (peptide) antibiotics (surfactin, iturin, bacillomycin, mycosubtilin, corynebactin/bacillibactin, fengycin plipastatin, mycobacillin, TL-119, bacilysin, bacilysocin, amicoumacin, 3,3′-neotrehalosadiamine, difficidin, rhizocticin).

For producing a fucosylated oligosaccharide, it is preferred to use a Bacillus cell that does not produce an antibiotic. Thus, in an additional and/or alternative embodiment, the Bacillus cell does not synthesize one or more of the antibiotics selected from the group consisting of lantibiotics and lantibiotic-like peptides such as subtilin, ericin S, mersacidin, sublancin 168, subtilosin A; non-ribosomally synthesized (peptide) antibiotics such as surfactin, iturin, bacillomycin, mycosubtilin, corynebactin/bacillibactin, fengycin plipastatin, mycobacillin, TL-119, bacilysin, bacilysocin, amicoumacin, 3,3′-neotrehalosadiamine, difficidin and rhizocticin. The Bacillus cell may have been genetically engineered to obtain a Bacillus cell that does not synthesize one or more of said antibiotics.

Wild-type Bacillus cells are capable of forming spores. Sporulation, i.e. the process of forming spores, in bacteria is considered a reaction of the bacterial cell which initiates a developmental program leading to the formation of daughter cells of distinct morphology and fate. Sporulation of Bacillus was studied as a basic model for cell differentiation. During sporulation, a rod-shaped Bacillus cell divides asymmetrically, thereby resulting in two genetically identical cells with different morphology and fates.

However, in industrial production it is not desired if the bacterial production strain sporulates during fermentation. Therefore, it is preferred to use Bacillus cells for the production of fucosylated oligosaccharides that are not capable of forming spores. Such Bacillus cells are called “non-sporulating”.

Preferably, the non-sporulating Bacillus cell capable to produce a fucosylated oligosaccharide originated from one of the B. subtilis strains listed in Table 1.

TABLE 1
Non-limiting list of Bacillus subtilis strains.

| Strain | Manufacturer/Reference | Product | Application |
|---|---|---|---|
| Bacillus subtilis 168 | BGSCID 1A1 | — | reference strain |
| Bacillus subtilis 168 | BGSCID BKE15310 | — | reference strain |
| ΔspollGA::erm | | | |
| Bacillus subtilis 168 | BGSCID BKE15320 | — | reference strain |
| ΔsigE::erm | | | |
| Bacillus subtilis 168 | BGSCID BKE15330 | — | reference strain |
| ΔsigG::erm | | | |
| Bacillus subtilis 168 | BGSCID BKE23450 | — | reference strain |
| ΔsigF::erm | | | |
| Bacillus subtilis 3NA | BGSCID 1S1 | — | reference strain |
| Bacillus subtilis W23 | BGSCID 2A9 | — | reference strain |
| Bacillus subtilis PY79 | BGSCID 1A747 | — | reference strain |
| Bacillus subtilis | Garden of Life ® | Primal Defense ® | Human use |
| Bacillus subtilis | Roux-Ocefa Laboratorios | Totalflora | Human use |
| Bacillus subtilis | Ist. Bioch. Italiano | Lactipan Plus | Human use |
| Bacillus subtilis 2335 | Biopharma | Biosporin ® | Human use |
| LifeinU ™ Bacillus subtilis | Lesaffre Human Care | Super Smart ® | Human use |
| CU1 | | | |
| Bacillus subtilis PXN/21 | Probiotics International Ltd | Bio-Kult ® | Human use |
| Bacillus subtilis HU58 | Microbiome LABS | HU58 ™ | Human use |
| Bacillus subtilis R0179 | Hanmi Pharmaceutical Co. | Medilac ® | Human use |
| | Ltd. | | |
| Bacillus subtilis DE111 | Deerland Enzymes & | NutriCommit | Human use |
| | Probiotics | | |
| Bacillus subtilis DSM21097 | MorinagaGarden B.V. | Morinaga Plus⁺ | Human use |
| Bacillus subtilis IDCC1101 | IIdong Pharmaceutical | Biobaby ® | Human use |
| Bacillus subtilis | JSL Foods | YoguFarm | Human use |
| KATMIRA1933 | | | |
| Bacillus subtilis spp. Natto | various | Natto | Human use |
| Bacillus subtilis SC-8 | various | Cheonggukjang | Human use |
| Bacillus subtilis | Christian Hansen | BioPlus2B ® | Veterinary Use |
| CH201/DSM5750 | | | |
| Bacillus subtilis PB6 | Kenim ® | CLOSTAT ® | Veterinary Use |

In an additional and/or alternative embodiment, the Bacillus cell has been genetically engineered by deletion or functional inactivation of Spo0A. Suitable functional inactivation of Spo0A includes deletion of the phosphorylation site, where Spo0F and Spo0B phosphotransferases phosphorylate Spo0A.

In an additional and/or alternative embodiment, the Bacillus cell has been genetically engineered by deletion or functional inactivation of the genes encoding sigma factor SigE (sigE) and/or sigma factor SigF (sigF).

The Bacillus cell of the invention is able to produce a fucosylated oligosaccharide when cultivated in the presence of lactose in a medium and under conditions that are permissive for the Bacillus cell to produce the fucosylated oligosaccharide.

According to the second aspect, provided is the use of a Bacillus cell as described herein before for the production of a fucosylated oligosaccharide, preferably a fucosylated human milk oligosaccharide such as 2′-FL, 3-FL or 2′,3-DiFL.

Since Bacillus is generally recognized as safe, production of fucosylated oligosaccharides for human consumption utilizing a production organism that is considered as generally safe should be considered safe too, provided that the amendments to the Bacillus cells for producing the fucosylated oligosaccharide does not affect the safety of the production strain with respect to human health and the environment. Hence, it is expected that there will be less concerns affecting regulatory approval of a fucosylated oligosaccharide for human consumption, in particular of fucosylated human milk oligosaccharides for human consumption, more specifically for infant formula, which consists of or contains a fucosylated oligosaccharide that has been produced by using a Bacillus cell as described herein. Thus, acceptance of microbially produced fucosylated oligosaccharides in infant formula and nutritional compositions at regulatory authorities as well as among consumers should be better.

According to the third aspect, provided is a method for the production of a fucosylated oligosaccharide the method comprises:
providing a non-sporulating Bacillus cell that had been genetically engineered to possess a lactose permease, a GDP-fucose biosynthesis pathway, and a fucosyltransferase;
cultivating the Bacillus cell in a culture medium containing lactose and under conditions that are permissive for the production of the fucosylated oligosaccharide, and
optionally retrieving the fucosylated oligosaccharide form the culture medium and/or from the Bacillus cell.

The non-sporulating Bacillus cell that is provided is a Bacillus cell as described herein.

In an additional and/or alternative embodiment, the fucosylated oligosaccharide is selected from the group consisting of 2′-fucosyllactose (2′-FL), 3-fucosyllactose (3-FL), 2′,3-difucosyllactose (DiFL), lacto-N-fucopentaose I (LNFP I), lacto-N-neofucopentaose I (LNnFP I), lacto-N-fucopentaose II (LNFP II), lacto-N-fucopentaose III (LNFP III), lacto-N-fucopentaose V (LNFP V), lacto-N-neofucopentaose V (LNnFP V), lacto-N-difucohexaose I (LNDH I) and lacto-N-difucohexaose II (LND).

In an additional and/or alternative embodiment, the culture medium contains L-fucose, in particular for cultivating a Bacillus cell for the production of a fucosylated oligosaccharide which possesses the GDP-fucose salvage pathway for providing GDP-fucose as donor substrate for the fucosyltransferase.

The invention also extends to fucosylated oligosaccharides that were produced by a Bacillus cell and/or a method as described herein, to the use of said fucosylated oligosaccharides for the manufacturing of a nutritional composition, preferably an infant formula, a dietary supplement or a medicinal food. Moreover, the present invention also extends to nutritional compositions containing a fucosylated oligosaccharide that was produced by a Bacillus cell and/or a method as described herein.

The present invention will be described with respect to particular embodiments and with reference to drawings, but the invention is not limited thereto but only by the claims. Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the invention, various features of the invention are sometimes grouped together in a single embodiment, FIGURE, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description and drawings provided herein, numerous specific details are set forth. However, it is understood that embodiments of the invention may be practiced without these specific details. In other instances, well-known methods, structures and techniques have not been shown in detail in order not to obscure an understanding. The invention will now be described by a detailed description of several embodiments of the invention. It is clear that other embodiments of the invention can be configured according to the knowledge of persons skilled in the art without departing from the true spirit or technical teaching of the invention, the invention being limited only by the terms of the appended claims.

### EXAMPLES

### Example 1: Transformation of Bacillus subtilis

Bacillus subtilis can be genetically manipulated by various techniques. For transformation of B. subtilis, competent cells were prepared by a modified protocol of the two-step method (Anagnostopoulos, C. and Spizizen, J. (1961) J Bacteriol 81 (5): 741-746). An overnight culture was inoculated in MG1 medium and shaken at 37° C. MG1 medium is Spizizen's minimal medium, which is supplemented with 0.5% glucose, 5 mM MgSO₄ and 0.02% casamino acids (optionally additionally supplemented with biotin and/or L-tryptophan). At the next morning, this culture was diluted 1:20 in fresh MG1 medium and incubated at 37° C. for approximately 6 h.

1 ml of the culture was diluted in 8 ml MG2 medium which differs from MG1 medium in the concentration of casamino acids (0.01% instead of 0.02%). In a shortened protocol the overnight culture is directly diluted in MG2 medium. After incubation for another 90 min, a 1-ml portion of the culture was mixed with 1-3 μg multimeric plasmid DNA or linear DNA and incubated at 37° C. for 30-60 min with shaking. Multimeric plasmid DNA was obtained either by using E. coli strain NM538 for propagation of plasmid DNA or by linearization of the plasmid by digestion with a single-cutter restriction enzyme, which cleaves within the backbone, followed by re-ligation with T4 DNA ligase.

Afterwards, cells were spread on 2×YT agar plates containing the appropriate antibiotic. Antibiotics were added in the following concentrations: 5 μg·mL⁻¹ erythromycin, 5 μg·mL⁻¹ chloramphenicol, 10 μg·mL⁻¹ kanamycin, 100 μg·mL⁻¹ spectinomycin.

Alternatively, for protoplast transformation (Romero, D. et al. (2006) Journal of Microbiological Methods 66:556-559), cells were grown in 20 ml of Penassay broth (PAB) at 37° C. until the onset of the stationary phase of growth (OD₆₀₀=1.7-2). Cells were then pelleted and resuspended in 10 ml of SMPP medium (0.3% bovine serum albumin, 5% 2 M sucrose, 25% 4×PAB, 50% 2×SMM), composition of 2×SMM being 1 M sucrose, 0.04 M maleic acid disodium salt hydrate and 0.04 M MgCl₂ (pH 6.5). After the addition of lysozyme (10 mg ml⁻¹) and mutanolysin (75 U ml⁻¹) the mixture was incubated at 37° C. with shaking to generate protoplasts. Protoplast formation was checked microscopically. Protoplasts were then carefully harvested by centrifugation at 5200×g and 4° C. for 5 min, washed twice with ice cold washing-electrotransformation buffer (1×SMM) and finally suspended in this solution. Plasmid DNA (1-3 μg) was added to 120 μl of protoplast suspension and the mixture was kept on ice for at least 5 min. The transformation mixture was transferred to a 0.2-cm cuvette and a single electroporation pulse was applied at 25 ρF, 400Ω and 0.7 kV. Immediately after the electroporation shock 1 ml of recovering medium (equal volumes of 4×PAB and 2×SMM, prepared freshly before use) was added to the cuvette. The transformation reaction was then transferred to a 2 ml tube and incubated at 37° C. with shaking for 12 h. For regeneration, the cell suspension was spread on DM3 agar plates (Chang, S. and Cohen, S. (1979) MGG 168(1):111-115) and incubated at 37° C. for 48 h. DM3 regeneration medium contained the following sterile solutions per liter: 200 ml 4% agar, 100 ml 5% casamino acids, 50 ml 10% yeast extract, 100 ml 3.5% K₂HPO₄ and 1.5% KH₂PO₄, 25 ml 20% glucose, 20 ml 1 M MgCl₂, 500 ml 0.5 M sorbitol and 5 ml filter sterilized 2% bovine serum albumin (added to the mixture when the temperature is below 55° C.) and was supplemented with the appropriate antibiotic.

Electroporation of B. subtilis was done according to a modified protocol from Zhang et al. (2011) provided by MoBiTec GmbH (Zhang, G., Bao, P., Zhang, Y., Deng, A., Chen, N. and Wen, T. (2011) Anal. Biochem., 409:130-137). A 2×YT overnight culture was diluted 100-fold with fresh 2×YT medium and the culture was grown to an OD₆₀₀ of 0.2 at 37° C. on a rotary shaker. Then the culture was supplemented with 1% DL-threonine, 2% glycine, 0.1% tryptophan and 0.03% Tween 80. After cultivation for another 60 min, the cell suspension was cooled on ice for 20 min, centrifuged at 5000×g for 10 min at 4° C. and washed twice with electroporation buffer (0.5 M trehalose, 0.5 M sorbitol, 0.5 M mannitol, 0.5 mM MgCl₂, 0.5 mM K₂HPO₄, 0.5 mM KH₂PO₄, pH 7.4, filter-sterilized and stored frozen). Finally, cells were resuspended in electroporation buffer at 1/100 of the original culture volume and 100 μl of cell suspension was mixed with DNA. The transformation mixture was transferred to a 0.1-cm cuvette and electroporation was performed at 1.8 kV with a single pulse delivered with a MicroPulser^{T}M device (Bio-Rad). Immediately after pulse delivery, 1 ml 2×YT broth containing 0.5 M sorbitol and 0.38 M mannitol was added to the cuvette. The transformation suspension was transferred to a 2 ml tube and incubated at 37° C. for 3 h on a rotary shaker. The cells were spread on a selective 2×YT agar plate and incubated at 37° C. overnight.

Using an alternative electroporation protocol (Xue, G. P., J. S. Johnson, and B. P. Dalrymple: 1999; Journal of Microbiological Methods 34:183-191), 5 ml of LB containing 0.5 M glucitol was inoculated with B. subtilis and incubated overnight at 37° C. Subsequently, the overnight culture was diluted (1:16) by 75 ml of LB containing 0.5 M glucitol and incubated until an OD₆₀₀ of 0.85-0.95 was obtained. The cells were then pelleted by centrifugation for 10 min at 4° C. at 5.000×g and washed four times by ice-cooled electroporation buffer (10% glycerol, 0.5 M glucitol, 0.5 M mannitol). Finally, the cells were resuspended in 1-2 ml electroporation buffer. Electroporation was performed using 60 μl of competent cells with DNA in a cooled electroporation cuvette (1-mm electrode gap). The cell-DNA mixture was subjected to a single electrical pulse at 25 ρF, 200Ω and 21 kV/cm. Finally, 1 ml recovery broth (LB containing 0.5 M glucitol and 0.38 mannitol) was added to the electropermeabilized cells and the bacterial culture was incubated for 3 h at 37° C. followed by plating on the LB agar supplemented by an antibiotic.

Two different rich media were used, namely Luria Broth (LB) and 2×YT.

Luria Broth (LB) medium consisted of 1% tryptone, 0.5% yeast extract and 0.5% NaCl (pH 7.2).

2×YT medium consisted of 1.6% tryptone, 1% yeast extract and 0.5% NaCl (pH 7.5).

To prepare rich-medium agar plates 15 g L⁻¹ agar were added.

For shaking flask experiments, Spizizen's minimal medium (Spizizen, J. 1958 Proc. Natl. Acad. Sci. U.S.A. 44(10):1072-1078) was used.

Spizizen's minimal medium contains the following salts: 2 g/L (NH₄)₂SO₄, 14 g/L K₂HPO₄, 6 g/L KH₂PO₄, 1 g/L Na₃ citrate×2.H₂O and 0.2 g/L MgSO₄×7.H₂O.

The preculture medium consisted of Spizizen's minimal salts supplemented with 2% D-glucose, 0.05% casamino acids and MgSO₄ to a final concentration of 2 mM (optionally additionally supplemented with biotin and/or L-tryptophan).

The main culture medium consisted of Spizizen's minimal salts supplemented with 2% D-glucose, 0.05% casamino acids, MgSO₄ to a final concentration of 2 mM and 0.5 mL·L⁻¹ 1000× trace element solution (optionally additionally supplemented with biotin and/or L-tryptophan).

Trace element solution (1000×) consisted of 100.6 g L⁻¹ C₆H₉NO₆, 56.4 g·L⁻¹ ammonium ferric citrate, 9.8 g·L⁻¹ MnCl₂×4.H₂O, 1.6 g·L⁻¹ COCl₂×6.H₂O, 1 g·L⁻¹ CuCl₂×2.H₂O, 1.9 g·L⁻¹ H₃BO₃, 9 g·L⁻¹ ZnSO₄×7.H₂O, 1.1 g·L⁻¹ Na₂MoO₄×2.H₂O, 1.5 g·L⁻¹ Na₂SeO₃, 1.5 g·L⁻¹ NiSO₄×6.H₂O.

If necessary, the appropriate antibiotic(s) were added to the medium to make it selective.

B. subtilis strains were initially grown on rich-media agar plates to obtain single colonies. These plates were grown for 1 day at 30-37° C. For shaking flask experiments, a 20-ml preculture was inoculated with a single colony and grown over night at 30-37° C. on a rotary shaker. Subsequent 20-ml main cultures were inoculated with this preculture to a starting OD₆₀₀ of about 0.1 and incubated at 30-37° C. on a rotary shaker. If induction was required, a 40-60 ml main culture was splitted into 20-ml proportions at the timepoint of induction. The culture volume did not exceed 20% of the shaking flask capacity.

### Example 2: Construction of a Bacillus subtilis Production Strain for 2′-Fucosyllactose

Metabolic engineering of a sporulation-deficient Bacillus subtilis strain (table 1) was achieved by the integration of the heterologous genes E. coli manC, E. coli manB and E. coli manA and simultaneous deletion of the endogenous gene lacA by homologous recombination. The B. subtilis gene ganA (yvfN, lacA), which is positioned within the galactan operon, encodes a beta-galactosidase.

For synthesis of GDP-mannose the open reading frames of manC, manB and manA were operably linked to the B. subtilis constitutive promoter P43 (iGem part repository: sequence ID: BBa_K143013) as an operon. The gene manC (Gen Bank accession number: NP_416553.1) encodes GDP:mannose-1-phosphate guanylyltransferase from E. coli. The gene manB (Gen Bank accession number: NP_416552.1) encodes E. coli phosphomannomutase and the gene manA (Gen Bank accession number: NP_389084.1) encodes B. subtilis fructose-6-phosphate isomerase. Each of the genes mentioned above was fused in silico to a B. subtilis RBS sequence. The herein described expression cassette <P43-manCBA> was codon-optimized for expression in B. subtilis and prepared synthetically by GenScript Corp. Subsequently, the complete integration cassette was assembled and cloned into pBR322 (New England Biolabs GmbH, Frankfurt, Germany) to create the suicide plasmid <pBR322 flank ganA up-lox71-erm-lox66-P43-manCBA-flank ganA down> (SEQ ID NO: 1). Afterwards, B. subtilis was transformed with this plasmid by its natural competence. Cells were spread on 2×YT agar plates containing the appropriate antibiotic (5 μg mL⁻¹ erythromycin). Integration of the expression cassette <P43-manCBA> into the ganA locus of the B. subtilis genome, yielding strain A, was verified by colony PCR. Gene expression was confirmed by targeted proteomics and/or by real-time PCR.

For the production of 2′-fucosyllactose from lactose and GDP-mannose in the resulting <P43-manCBA> integration strain, an expression plasmid (SEQ ID NO: 2) was constructed that comprises all necessary genes under the control of the inducible promoter P_{grac100}. Therefore, the B. subtilis expression vector pHT253 (MoBiTec GmbH, Göttingen, Germany) was used as backbone. The open reading frame of the E. coli lacY gene (Gen Bank accession number: NP_414877.1) was amplified by PCR from chromosomal DNA. The open reading frame of the gene wbgL, encoding for a E. coli O126 α-1,2-fucosyltransferases, was codon-optimized for expression in B. subtilis and prepared synthetically by GenScript Corp. Also, the open reading frames of the E. coli genes gmd (Gen Bank accession number: NP_416557.1; encoding for a GDP-mannose-4,6-dehydratase) and wcaG (Gen Bank accession number: NP_416556.1; encoding for a GDP-fucose synthase) were codon-optimized for expression in B. subtilis and prepared synthetically by GenScript Corp. Each gene enclosed in the inducible expression cassette was linked to a B. subtilis RBS sequence. Additionally, a suitable B. subtilis terminator sequence from the iGem part repository (sequence ID: BBa_B0015) was introduced in the final expression plasmid <pHT253 P_{grac100}-wbgL-gmd-wcaG-lacY-terminator> (SEQ ID NO: 2). The <P43-manCBA> integration strain was transformed with this expression plasmid (SEQ ID NO: 2) by its natural competence. Gene expression was confirmed by targeted proteomics and/or by real-time PCR. Transformants were cultivated under conditions that are permissive for the B. subtilis to produce 2′-fucosyllactose in the presence of exogenous lactose.

### Example 3: Construction of a Bacillus subtilis Production Strain for 2′-Fucosyllactose

Strain A, which is described in example 2, was used as parent strain. For the production of 2′-fucosyllactose from lactose and GDP-mannose, additionally, the E. coli genes lacY, gmd, wcaG and wbgL were integrated in the endogenous amyE (amyA) locus of strain A (encoding for an alpha-amylase).

The open reading frame of the gene wbgL (encoding for a α-1,2-fucosyltransferase) was codon-optimized for expression in B. subtilis and prepared synthetically by GenScript Corp. Also, the open reading frames of the genes gmd (Gen Bank accession number: NP_416557.1; encoding for a GDP-mannose-4,6-dehydratase) and wcaG (Gen Bank accession number: NP_416556.1; encoding for a GDP-fucose synthase) were codon-optimized for expression in B. subtilis (carried out by GenScript Corp), placed under the control of the strong constitutive B. subtilis promoter P_{lepA} as a two-gene operon and prepared synthetically by GenScript Corp. The open reading frame of the E. coli lacY gene (Gen Bank accession number: NP_414877.1), encoding a lactose permease, was amplified by PCR from chromosomal DNA.

All necessary genes for the production of 2′-fucosyllactose from lactose and GDP-mannose were assembled to the constitutive expression cassette <P43-wbgL-lacY-P_{lepA}-gmd-wcaG> in which each gene was linked to a B. subtilis RBS sequence. Additionally, a suitable B. subtilis terminator sequence from the iGem part repository (sequence ID: BBa_B0015) was placed downstream of this expression cassette. The above-described construct was cloned into pBR322 (New England Biolabs GmbH, Frankfurt, Germany) to generate <pBR322 flank amyE up-lox71-aad9-lox66-P43-wbgL-lacY-P_{lepA}-gmd wcaG-terminator-flank amyE down> (SEQ ID NO: 3).

The resulting amyE integration vector (SEQ ID NO: 3) was used to transform B. subtilis by its natural competence. Transformants were selected by spectinomycin (100 μg ml⁻¹). Integration of the expression cassette <P43-wbgL-lacY-P_{lepA}-gmd wcaG-terminator> into the amyE locus of the B. subtilis genome was verified by colony PCR. Gene expression was confirmed by targeted proteomics and/or by real-time PCR.

### Example 4: Construction of a Bacillus subtilis Production Strain for 2′-Fucosyllactose

For the production of 2′-fucosyllactose from GDP-fucose and exogenous lactose, initially, the E. coli gene lacY was integrated into the endogenous amyE (amyA) locus (encoding for an alpha-amylase). The open reading frame of the E. coli lacY gene (Gen Bank accession number: NP_414877.1), encoding a lactose permease, was amplified by PCR from chromosomal DNA. The integration cassette <flank amyE up-lox71-aad9-lox66-P43-lacY-flank amyE down> (SEQ ID NO: 4) was constructed and cloned into pBR322 (New England Biolabs GmbH, Frankfurt, Germany). B. subtilis was transformed with the resulting plasmid by its natural competence. Integration of the expression cassette <P43-lacY> into the amyE locus of the B. subtilis genome, yielding strain B, was verified by colony PCR. Gene expression was confirmed by targeted proteomics and/or by real-time PCR.

For synthesis of GDP-fucose from exogenous L-fucose a B. subtilis expression plasmid was constructed, comprising all necessary genes. Plasmid pDW1 was used as backbone. This plasmid was prepared synthetically by GenScript Corp and contains the genes bla and erm for selection in E. coli and B. subtilis, respectively. Additionally, it contains the replicon of pMB1 for propagation in E. coli and the pUB110 replicon for propagation in B. subtilis.

The B. fragilis gene fkp (GenBank accession number AY849806) encodes for a bifunctional fucose kinase/L-fucose-1-phosphate guanylyltransferase, the E. coli gene wbgL encodes for a α-1,2-fucosyltransferases and the E. coli gene fucP (Gen Bank accession number: NP_417281.1) encodes for a L-fucose permease. The open reading frames of these genes were each codon-optimized for expression in B. subtilis and prepared synthetically by GenScript Corp. An expression cassette was constructed that comprises the genes fkp, wbgL and fucP under the control of the strong constitutive B. subtilis promoter P43.

Each gene enclosed in the expression cassette was linked to a B. subtilis RBS sequence. Additionally, a suitable B. subtilis terminator sequence from the iGem part repository (sequence ID: BBa_B0015) was introduced in the final expression plasmid <pDW1-P43-fkp-wbgL-fucP-terminator> (SEQ ID NO: 5). Strain B was transformed with this expression plasmid (SEQ ID NO: 5) by its natural competence. Gene expression was confirmed by targeted proteomics and/or by real-time PCR. Transformants were cultivated under conditions that are permissive for B. subtilis to produce 2′-fucosyllactose in the presence of exogenous lactose and L-fucose.

### Example 5: Production of 2′-Fucosyllactose Using Metabolically Engineered Bacillus subtilis Strains

A preculture was inoculated with a metabolically engineered Bacillus subtilis strain suitable for the biosynthesis of 2′-fucosyllactose (as described in example 2, 3 and 4, respectively).

The preculture was incubated at 30-37° C. over night and then diluted to a starting OD₆₀₀ of about 0.1 in fresh main culture medium. When the main culture reached an OD₆₀₀ of approximately 0.5, 2 mM lactose was added to the growth medium. When the inducible promoter P_{grac100} was used for gene expression, induction was carried out with lactose (2 mM) or with both lactose (2 mM) and IPTG (1 mM). For the salvage biosynthesis of GDP-fucose, additionally, 2 mM L-fucose were added. Cultivation was discontinued after 24 h/48 h and intracellular and extracellular 2′-fucosyllactose was analyzed by thin layer chromatography and/or HPLC and/or mass spectrometry (as described in WO 2017/042382 A or WO 2019/008133 A). Biosynthesis of substantial amounts of 2′-fucosyllactose was detected.

1. A non-sporulating Bacillus cell for the production of a fucosylated oligosaccharide, wherein said cell has been genetically engineered to possess a lactose permease, a GDP-fucose biosynthesis pathway and a fucosyltransferase.

2. The Bacillus cell according to claim 1, wherein the sporulation has been impaired by deletion or functional inactivation of one or more of the genes encoding Spo0A, sigma E and sigma F.

3. The Bacillus cell according to claim 1, wherein the lactose permease is E. coli LacY or a functional variant thereof.

4. The Bacillus cell according to claim 1, wherein the GDP-fucose biosynthesis pathway is a de novo GDP-fucose biosynthesis pathway and/or a salvage pathway.

5. The Bacillus cell according to claim 1, wherein the fucosyltransferase is a lactose-accepting fucosyltransferase, optionally a fucosyltransferase selected from the group of α-1,2-fucosyltransferases and α-1,3-fucosoyltransferases.

6. The Bacillus cell according to claim 1, wherein said Bacillus cell lacks any β-galactosidase activity or possesses reduced β-galactosidase activity as compared to a wild-type progenitor Bacillus cell of the same species.

7. The Bacillus cell according to claim 6, wherein the Bacillus cell has been genetically engineered by deletion or functional inactivation of at least one of the genes selected from the group consisting of yesZ and ganA.

8. The Bacillus cell according to claim 1, wherein said Bacillus cell is a Bacillus subtilis cell.

9. A product comprising a Bacillus cell according to claim 1 for production of a fucosylated oligosaccharide.

10. A method for production of a fucosylated oligosaccharide, comprising
providing a non-sporulating Bacillus cell as defined in claim 1;
cultivating said Bacillus cell in a medium and under conditions that are permissive for production of a fucosylated oligosaccharide; and optionally
retrieving the fucosylated oligosaccharide from the medium and/or the Bacillus cell.

11. The method according to claim 10, wherein the medium comprises lactose.

12. The method according to claim 10, wherein the medium comprises L-fucose.

13. A fucosylated oligosaccharide produced by a method according to claim 10.

14. A product comprising a fucosylated oligosaccharide according to claim 13 for manufacture of a nutritional composition, optionally of an infant formula.

15. A nutritional composition comprising a fucosylated oligosaccharide according to claim 13.
